(19) United States
(12) Patent Application Publication
(10) Pub. No.: US 2026/0047765 A1
(43) Pub. Date: Feb. 19, 2026

(54) Fiber Optics Thermometry System for Detection and Confirmation

(71) Applicant: Bard Access Systems, Inc., Salt Lake City, UT (US)

(72) Inventors: Anthony K. Misener, Bountiful, UT (US); Steffan Sowards, Salt Lake City, UT (US); William Robert McLaughlin, Bountiful, UT (US)

(21) Appl. No.: 18/808,868

(22) Filed: Aug. 19, 2024

(51) Int. Cl. A61B 5/01; A61B 5/00; A61B 5/1455; A61B 5/20

(52) U.S. Cl. CPC A61B 5/015; A61B 5/0075; A61B 5/0084; A61B 5/202; A61B 5/1455

(57) ABSTRACT

A system, apparatus and method directed to determining a temperature within a patient body, including an optical fiber with one or more core fibers. The system can include a console having non-transitory computer-readable medium storing logic that, when executed, causes operations of providing an incident light signal to the optical fiber, receiving a reflected light signal of the incident light, processing the reflected light signal to determine a temperature within the patient body near a measurement region. The method may include determining a location of a distal tip of the optical fiber within the patient body at least based on the temperature.

### BACKGROUND

In the past, certain intravascular guidance of medical devices, such as guidewires and catheters for example, have used fluoroscopic methods for tracking tips of the medical devices and determining whether distal tips are appropriately localized in their target anatomical structures. However, such fluoroscopic methods expose patients and their attending clinicians to harmful X-ray radiation. Moreover, in some cases, the patients are exposed to potentially harmful contrast media needed for the fluoroscopic methods.

More recently, electromagnetic tracking systems have been used involving stylets. Generally, electromagnetic tracking systems feature three components: a field generator, a sensor unit and control unit. The field generator uses several coils to generate a position-varying magnetic field, which is used to establish a coordinate space. Attached to the stylet, such as near a distal end (tip) of the stylet for example, the sensor unit includes small coils in which current is induced via the magnetic field. Based on the electrical properties of each coil, the position and orientation of the medical device may be determined within the coordinate space. The control unit controls the field generator and captures data from the sensor unit.

Although electromagnetic tracking systems avoid line-of-sight reliance in tracking the tip of a stylet while obviating radiation exposure and potentially harmful contrast media associated with fluoroscopic methods, electromagnetic tracking systems are prone to interference. More specifically, since electromagnetic tracking systems depend on the measurement of magnetic fields produced by the field generator, these systems are subject to electromagnetic field interference, which may be caused by the presence of many different types of consumer electronics such as cellular telephones. Additionally, electromagnetic tracking systems are subject to signal drop out, depend on an external sensor, and are defined to a limited depth range.

Disclosed herein is a system including a medical instrument having an optical fiber disposed therein and methods performed thereby where the system is configured to provide confirmation of tip placement or tracking information using optical fiber technology. Further, the system is configured to detect temperatures within a body conduit of a patient.

Some embodiments combine the temperature detection functionality with one or more of a fiber optic shape sensing functionality, intravascular electrocardiogram (ECG) monitoring, impedance/conductance sensing, and oxygen detection functionality.

### SUMMARY

Briefly summarized, embodiments disclosed herein are directed to systems, apparatus and methods for obtaining thermometry data and, optionally, three-dimensional (3D) information corresponding to a trajectory and/or shape of a medical instrument, such as a catheter, a guidewire, or a stylet, via a fiber optic core during advancement through body conduits of a patient and assisting in navigation of the medical instrument during advancement.

More particularly, in some embodiments, the medical instrument includes one or more optical fiber cores, where each are configured with an array of sensors (reflective gratings), which are spatially distributed over a prescribed length of the core fiber to generally sense external strain and temperature on those regions of the core fiber occupied by the sensor. Each optical fiber core is configured to receive light (e.g., broadband light, infrared light, near infrared light, etc.) from a console during advancement through body conduits of a patient, where the light propagates along at least a partial distance of the optical fiber core toward the distal end. For purposes of clarity, the terms incident light or broadband incident light may be utilized in the description below; however, infrared light and near infrared light may be alternatively utilized. Given that each sensor positioned along the optical fiber core is configured to reflect light of a different, specific spectral width, the array of sensors enables distributed measurements throughout the prescribed length of the medical instrument. These distributed measurements may include wavelength shifts having a correlation with strain and/or temperature experienced by the sensor.

The reflected light from the sensors (reflective gratings) within an optical fiber core is returned from the medical instrument for processing by the console. The physical state of the medical instrument may be ascertained based on analytics of the wavelength shifts of the reflected light. For example, the strain caused through bending of the medical instrument and hence angular modification of the optical fiber core, causes different degrees of deformation. The different degrees of deformation alter the shape of the sensors (reflective grating) positioned on the optical fiber core, which may cause variations (shifts) in the wavelength of the reflected light from the sensors positioned on the optical fiber core. The optical fiber core may comprise a single optical fiber, or a plurality of optical fibers (in which case, the optical fiber core is referred to as a “multi-core optical fiber”).

As used herein, the term “core fiber,” generally refers to a single optical fiber core disposed within a medical device. Thus, discussion of a core fiber refers to single optical fiber core and discussion of a multi-core optical fiber refers to a plurality of core fibers. Various embodiments discussed below to detection of the health (and particularly the damage) that occurs in each of an optical fiber core of medical instrument including (i) a single core fiber, and (ii) a plurality of core fibers. It is noted that in addition to strain altering the shape of a sensor, ambient temperature variations may also alter the shape of a sensor, thereby causing variations (shifts) in the wavelength of the reflected light from the sensors positioned on the optical fiber core.

More particularly, in some embodiments the optical fiber disposed within the medical instrument may be configured to act as a measurement system of temperature within a body conduit of a patient. Additionally, a measured temperature may indicate a particular location within the patient (e.g., a blood vessel has a temperature than the right atrium and arterial blood is warmer than venous blood). As incident light provided by the console propagates along at least a partial distance of the medical instrument toward a distal end, it can reach one or more sensors, which may be spatially distributed over a prescribed length of the medical instrument. Given that each sensor positioned along the optical fiber core is configured to reflect light of a different, specific spectral width, the one or more sensors can be configured to enable distributed measurements throughout the prescribed length of the medical instrument. These distributed measurements may include wavelength shifts having a correlation with temperature experienced by the one or more sensors. Specifically, the temperature at different regions of the medical instrument may be ascertained based on analytics of the wavelength shifts of the reflected light. Temperature within a patient body conduit alters the characteristics of the sensors (reflective grating) positioned on the optical fiber core, which may cause variations (shifts) in the wavelength of the reflected light from the sensors positioned on the optical fiber core. For example, higher temperatures may increase the refractive index and thermal expansion of the sensors leading to a positive wavelength shift in the reflected light. Conversely, lower temperatures may decrease the refractive index and thermal expansion of the sensors causing a negative wavelength shift in the reflected light.

Specifically, the reflected light from the one or more sensors subsequently propagates back to the console for processing. The console is configured to analyze the reflected light from one or more sensors by correlating the reflected light to a temperature at each sensor. In some embodiments, the light reflected by the one or more sensors is analyzed by logic of the console to determine a location of the distal tip of the optical fiber based on a comparison of the reflected light with data corresponding to known locations within a body conduit as the temperature varies at different locations of a patient's body.

In particular, the reflected light is received by an optical receiver of the console, which is configured to translate the reflected light signals into reflection data, namely data in the form of electrical signals representative of the reflected light signals. The logic of the console is configured to determine a correlation between the reflection data and temperature, where the logic may then corelate a temperature to a particular location of the distal tip of the optical fiber within the patient. In some embodiments, the site at which the optical fiber entered the patient may be utilized in determining the location within the patient. For instance, when two locations each closely correlate to the determined temperature, the logic of the console may select a particular location based on proximity to the entry site, and optionally knowledge of advancement of the distal tip of the optical fiber within the patient. For example, the logic of the console may select a location option based on temperature based on proximity to the entry site (e.g., a location option within the shoulder may be selected over a location option in the leg when the entry site of the optical fiber is the cephalic vein of a patient's forearm). Other embodiments utilizing the reflection data are discussed below that may also assist a clinician in navigating advancement of the optical fiber (and corresponding medical instrument).

Specific embodiments of the disclosure include utilization of a medical instrument, such as a stylet, featuring a multi-core optical fiber and a conductive medium that collectively operate for tracking placement with a body of a patient of the stylet or another medical device (such as a catheter) in which the stylet is disposed. In lieu of a stylet, a guidewire may be utilized. For convenience, embodiments are generally discussed where the optical fiber core is disposed within a stylet; however, the disclosure is not intended to be so limited as the functionality involving detection of the health of an optical fiber core disclosed herein may be implemented regardless of the medical device in which the optical fiber core is disposed. In some embodiments, the optical fiber core may be integrated directly into a wall of the catheter.

In some embodiments, the optical fiber core of a stylet is configured to return information for use in identifying its physical state (e.g., shape length, shape, and/or form) of (i) a portion of the stylet (e.g., tip, segment of stylet, etc.) or a portion of a catheter inclusive of at least a portion of the stylet (e.g., tip, segment of catheter, etc.) or (ii) the entirety or a substantial portion of the stylet or catheter within the body of a patient (hereinafter, described as the “physical state of the stylet” or the “physical state of the catheter”). According to one embodiment of the disclosure, the returned information may be obtained from reflected light signals of different spectral widths, where each reflected light signal corresponds to a portion of broadband incident light propagating along a core of the multi-core optical fiber (core fiber) that is reflected back over the core fiber by a particular sensor located on the core fiber. One illustrative example of the returned information may pertain to a change in signal characteristics of the reflected light signal returned from the sensor, where wavelength shift is correlated to (mechanical) strain on the core fiber or a detected change in ambient temperature.

In some embodiments, the core fiber utilizes a plurality of sensors, and each sensor is configured to reflect a different spectral range of the incident light (e.g., different light frequency range). Based on the type and degree of strain and/or temperature asserted on each core fiber, the sensors associated with that core fiber may alter (shift) the wavelength of the reflected light to convey the type and degree of strain and/or temperature on that core fiber at those locations of the stylet occupied by the sensors. The sensors are spatially distributed at various locations of the core fiber between a proximal end and a distal end of the stylet so that shape sensing of the stylet may be conducted based on analytics of the wavelength shifts. Herein, the shape sensing functionality is paired with the ability to simultaneously pass an electrical signal through the same member (stylet) through conductive medium included as part of the stylet.

More specifically, in some embodiments each core fiber of the multi-core optical fiber is configured with an array of sensors, which are spatially distributed over a prescribed length of the core fiber to generally sense external strain on or variations in ambient temperature proximate those regions of the core fiber occupied by the sensor. Given that each sensor positioned along the same core fiber is configured to reflect light of a different, specific spectral width, the array of sensors enables distributed measurements throughout the prescribed length of the multi-core optical fiber. These distributed measurements may include wavelength shifts having a correlation with strain experienced and/or temperature variations detected by the sensor.

In more detail, each sensor may operate as a reflective grating such as a fiber Bragg grating (FBG), namely an intrinsic sensor corresponding to a permanent, periodic refractive index change inscribed into the core fiber. Stated differently, the sensor operates as a light reflective mirror for a specific spectral width (e.g., a specific wavelength or specific range of wavelengths). As a result, as broadband incident light is supplied by an optical light source and propagates through a particular core fiber, upon reaching a first sensor of the distributed array of sensors for that core fiber, light of a prescribed spectral width associated with the first sensor is reflected back to an optical receiver within a console, including a display and the optical light source. The remaining spectrum of the incident light continues propagation through the core fiber toward a distal end of the stylet. The remaining spectrum of the incident light may encounter other sensors from the distributed array of sensors, where each of these sensors is fabricated to reflect light with different specific spectral widths to provide distributed measurements, as described above.

During operation, multiple light reflections (also referred to as “reflected light signals”) are returned to the console from each of the plurality of core fibers of the multi-core optical fiber. Each reflected light signal may be uniquely associated with a different spectral width. Information associated with the reflected light signals may be used to determine a three-dimensional representation of the physical state of the stylet within the body of a patient through detection of strain and/or an ambient temperature variation in response to emitted incident light. Herein, the core fibers are spatially separated with the cladding of the multi-mode optical fiber and each core fiber is configured to separately return light of different spectral widths (e.g., specific light wavelength or a range of light wavelengths) reflected from the distributed array of sensors fabricated in each of the core fibers.

During vasculature insertion and advancement of the catheter, the clinician may rely on the console to visualize a current physical state (e.g., shape) of a catheter guided by the stylet to avoid potential path deviations. As the periphery core fibers reside at spatially different locations within the cladding of the multi-mode optical fiber, changes in angular orientation (such as bending with respect to the center core fiber, etc.) of the stylet imposes different types (e.g., compression or tension) and degrees of strain on each of the periphery core fibers as well as the center core fiber. The different types and/or degree of strain may cause the sensors of the core fibers to apply different wavelength shifts, which can be measured to extrapolate the physical state of the stylet (catheter).

Certain embodiments of the disclosure pertain to the utilization of fiber optic shape sensing, detection of temperature to track advancement of a medical device throughout the vasculature of a patient. For example, as noted above, each core fiber includes a plurality of reflective gratings disposed along its length, wherein each reflective grating receives broadband incident light and reflects light signals having a specific spectral width (e.g., a specific wavelength or specific range of wavelengths) that may be shifted based on a temperature of a length of the core fiber corresponding to the reflective grating.

In some embodiments each of the one or more core fibers includes a single sensor disposed at a distal tip of the optical fiber configured to (i) reflect a light signal of a different, specific spectral width based on received incident light, and (ii) change a characteristic of the reflected light signal for use in determining the temperature of the optical fiber. In this embodiment, processing of the reflected light signal involves a comparison with a reference wavelength corresponding to a known external reference temperature.

In yet other embodiments, each of the one or more core fibers includes a plurality of sensors distributed along a longitudinal length of a corresponding core fiber and each sensor of the plurality of sensors is configured to (i) reflect a light signal of a different spectral width based on received incident light, and (ii) change a characteristic of the reflected light signal for use in determining a physical state of the optical fiber. In this embodiment, a temperature gradient display can be generated.

Some embodiments include a medical device system for determining a temperature within a patient body, where the system includes the medical instrument including an optical fiber having one or more of core fibers. The system may also include a console including one or more processors and a non-transitory computer-readable medium having stored thereon logic, when executed by the one or more processors, causes operations including providing an incident light signal to the optical fiber, receiving a reflected light signal of the incident light, and processing the reflected light signal to determine the temperature within the patient body.

In further embodiments, the logic, when executed by the one or more processors, causes further operations including generating a display indicating the location of the distal tip of the optical fiber within the patient body.

In some embodiments, the medical instrument is one of an introducer wire, a guidewire, a stylet, a stylet within a needle, a needle with the optical fiber inlayed into a cannula of the needle or a catheter with the optical fiber inlayed into one or more walls of the catheter.

Additionally, systems and methods disclosed may perform operations to determine whether the needle has been placed within a vein or an artery. Specifically, the logic of the console may determine that the reflection data correlates to a specific temperature, where such temperature corresponds to either a vein (e.g., lower temperature) or an artery (e.g., higher temperature).

Other embodiments of the disclosure are directed to a method of determining a temperature within a body of a patient. The method includes operations of providing an incident light signal to an optical fiber disposed within the medical instrument, wherein the optical fiber includes one or more core fibers, receiving a reflected light signal of the incident light, and processing the reflected light signal to determine temperature within the patient body.

In some embodiments, each of the one or more core fibers includes a plurality of sensors distributed along a longitudinal length of a corresponding core fiber and each sensor of the plurality of sensors is configured to (i) reflect a light signal of a different spectral width based on received incident light, and (ii) change a characteristic of the reflected light signal for use in determining a physical state of the optical fiber. In yet some embodiments, the optical fiber is a single-core optical fiber, and wherein the incident light is provided in pulses.

In some embodiments, the optical fiber is a multi-core optical fiber including a plurality of core fibers, and wherein the incident light propagates along a first core fiber and the reflect light signal propagates along a second core fiber. In other embodiments, determining the location of the distal tip of the optical fiber within the patient body is based on the temperature and an entry site of the medical instrument.

In further embodiments, the logic, when executed by the one or more processors, causes further operations including generating a display indicating the location of the distal tip of the optical fiber within the patient body.

In some embodiments, the medical instrument is one of an introducer wire, a guidewire, a stylet, a stylet within a needle, a needle with the optical fiber inlayed into a cannula of the needle or a catheter with the optical fiber inlayed into one or more walls of the catheter.

Yet other embodiments include a non-transitory computer-readable medium having stored thereon logic that, when executed by one or more processors, causes operations including providing an incident light signal to an optical fiber disposed within the medical instrument, the optical fiber including one or more core fibers, receiving a reflected light signal of the incident light, and processing the reflected light signal to determine a temperature within the patient body.

In some embodiments, each of the one or more core fibers includes a plurality of sensors distributed along a longitudinal length of a corresponding core fiber and each sensor of the plurality of sensors is configured to (i) reflect a light signal of a different spectral width based on received incident light, and (ii) change a characteristic of the reflected light signal for use in determining a physical state of the optical fiber. In yet some embodiments, the optical fiber is a single-core optical fiber, and wherein the incident light is provided in pulses.

In some embodiments, the optical fiber is a multi-core optical fiber including a plurality of core fibers, and wherein the incident light propagates along a first core fiber and the reflect light signal propagates along a second core fiber. In other embodiments, determining the location of the distal tip of the optical fiber within the patient body is based on the temperature and an entry site of the medical instrument.

In further embodiments, the logic, when executed by the one or more processors, causes further operations including generating a display indicating the location of the distal tip of the optical fiber within the patient body.

In some embodiments, the medical instrument is one of an introducer wire, a guidewire, a stylet, a stylet within a needle, a needle with the optical fiber inlayed into a cannula of the needle or a catheter with the optical fiber inlayed into one or more walls of the catheter.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1A is an illustrative embodiment of a medical instrument monitoring system including a medical instrument with optic shape sensing and fiber optic-based thermometry capabilities in accordance with some embodiments;

FIG. 1B is an alternative illustrative embodiment of the medical instrument monitoring system 100 in accordance with some embodiments;

FIG. 2 is an exemplary embodiment of a structure of a section of the multi-core optical fiber included within the stylet 120 of FIG. 1A in accordance with some embodiments;

FIG. 3A is a first exemplary embodiment of the stylet of FIG. 1A supporting both an optical and electrical signaling in accordance with some embodiments;

FIG. 3B is a cross sectional view of the stylet of FIG. 3A in accordance with some embodiments;

FIG. 4A is a second exemplary embodiment of the stylet of FIG. 1B in accordance with some embodiments;

FIG. 4B is a cross sectional view of the stylet of FIG. 4A in accordance with some embodiments;

FIG. 5A is an elevation view of a first illustrative embodiment of a catheter including integrated tubing, a diametrically disposed septum, and micro-lumens formed within the tubing and septum in accordance with some embodiments;

FIG. 5B is a perspective view of the first illustrative embodiment of the catheter of FIG. 5A including core fibers installed within the micro-lumens in accordance with some embodiments;

FIGS. 6A-6B are flowcharts of the methods of operations conducted by the medical instrument monitoring system of FIGS. 1A-1B to achieve optic 3D shape sensing in accordance with some embodiments;

FIG. 7 is an exemplary embodiment of the medical instrument monitoring system of FIG. 1A during operation and insertion of the catheter into a patient in accordance with some embodiments;

FIG. 8 is a cross sectional perspective view of a body conduit having a medical instrument advancing therein where the medical instrument includes an optical fiber having a single sensor disposed at a distal tip shown to be receiving and reflecting light in accordance with some embodiments;

FIG. 9 is a cross sectional perspective view of the blood vessel of FIG. 8 having the medical instrument advancing therein where the medical instrument includes an optical fiber having plurality of sensors distributed throughout a length of the fiber, wherein each sensor is shown to be receiving and reflecting light in accordance with some embodiments;

FIGS. 10A-10B are exemplary embodiments of the medical instrument monitoring system of FIGS. 1A-1B deploying the medical instrument and optical fiber of FIG. 9 within a bladder of a patient are shown in accordance with some embodiments;

FIG. 11 is an embodiment of medical instrument monitoring system of FIGS. 1A-1B deploying the medical instrument and optical fiber of FIG. 9 in a heart of a patient during an ablation procedure is shown in accordance with some embodiments;

FIG. 12 an exemplary embodiment of the medical instrument monitoring system of FIG. 1A during insertion and operation, is shown to be infusing a fluid in accordance with some embodiments;

FIG. 13 is a flowchart illustrating an exemplary method of operations conducted by the medical instrument monitoring system of either of FIGS. 1A-1B deploying the medical instrument and optical fiber of FIG. 8 to achieve optic temperature sensing is shown in accordance with some embodiments;

FIGS. 14A-14B are flowcharts illustrating exemplary methods of operations conducted by the medical instrument monitoring system of either of FIGS. 1A-1B deploying the medical instrument and optical fiber of FIG. 9 to achieve optic temperature sensing are shown in accordance with some embodiments;

FIG. 15 is a flowchart illustrating an embodiment of training and deploying a machine learning model to achieve optic temperature sensing in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to, a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random-access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

The term “body conduit” refers to any biological canal, vessel, or passageway within a body of a patient that facilitates the transport or movement of substances. This includes, but is not limited to, vasculature vessels (arteries, veins, and capillaries), lymphatic vessels, gastrointestinal tracts, airways (such as bronchi and bronchioles), urinary tracts, and other structures like ducts and sinuses that convey fluids, gases, or solids. Examples of such body conduits may further include organs or organ-specific pathways which include, but are not limited to, the heart, bladder, or lungs.

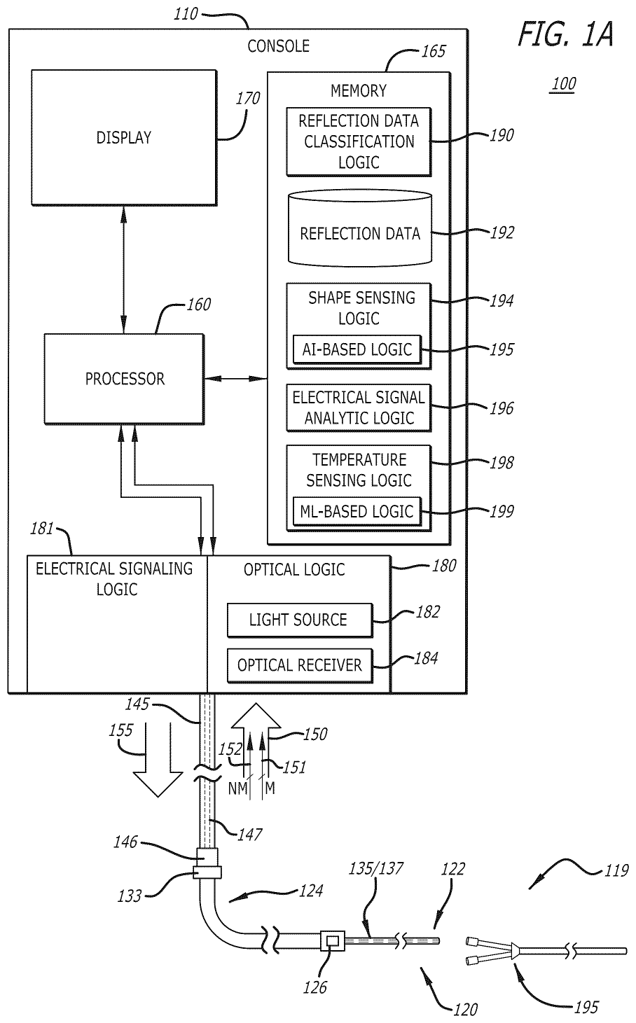

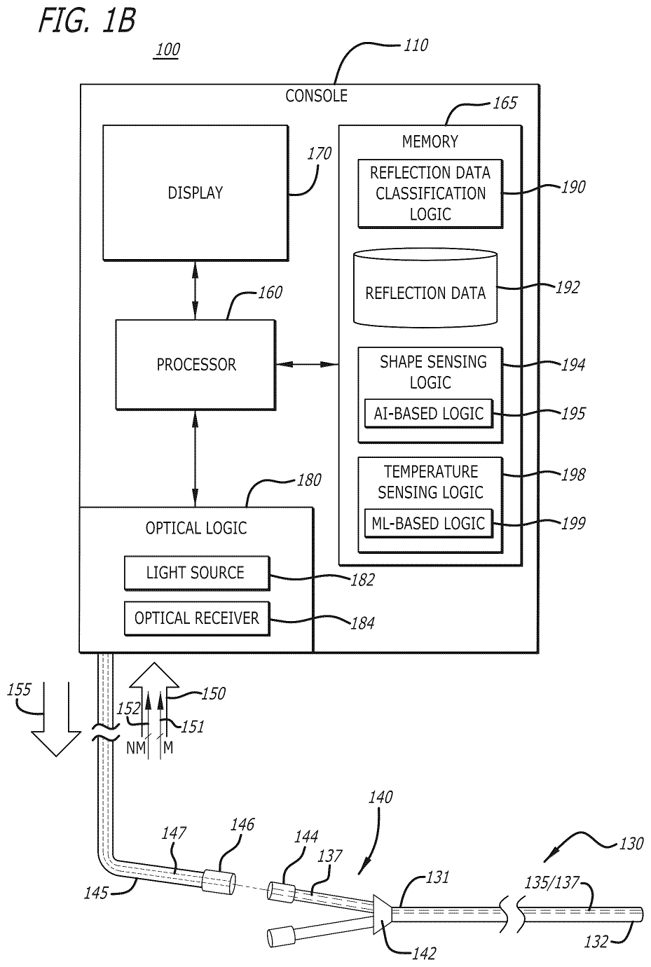

Referring to FIG. 1A, an illustrative embodiment of a medical instrument monitoring system including a medical instrument with optic shape sensing and fiber optic-based temperature sensing capabilities is shown in accordance with some embodiments. As shown, the system 100 generally includes a console 110 and a stylet assembly 119 communicatively coupled to the console 110. For this embodiment, the stylet assembly 119 includes an elongate probe (e.g., stylet) 120 on its distal end 122 and a console connector 133 on its proximal end 124, where the stylet 120 is configured to advance within a body conduit(s) of a patient either through, or in conjunction with, a catheter 195. The console connector 133 enables the stylet assembly 119 to be operably connected to the console 110 via an interconnect 145 including one or more optical fibers 147 (hereinafter, “optical fiber(s)”) and a conductive medium terminated by a single optical/electric connector 146 (or terminated by dual connectors). Herein, the connector 146 is configured to engage (mate) with the console connector 133 to allow for the propagation of light between the console 110 and the stylet assembly 119 as well as the propagation of electrical signals from the stylet 120 to the console 110.

An exemplary implementation of the console 110 includes a processor 160, a memory 165, a display 170 and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Pat. No. 10,992,078, the entire contents of which are incorporated by reference herein. The processor 160, with access to the memory 165 (e.g., non-volatile memory or non-transitory, computer-readable medium), is included to control functionality of the console 110 during operation. As shown, the display 170 may be a liquid crystal diode (LCD) display integrated into the console 110 and employed as a user interface to display information to the clinician, especially during a catheter placement procedure (e.g., cardiac catheterization). In another embodiment, the display 170 may be separate from the console 110. Although not shown, a user interface is configured to provide user control of the console 110.

For both embodiments, the content depicted by the display 170 may change according to which mode the stylet 120 is configured to operate: optical, TLS, ECG, or another modality. In TLS mode, the content rendered by the display 170 may constitute a two-dimensional (2D) or three-dimensional (3D) representation of the physical state (e.g., length, shape, form, and/or orientation) of the stylet 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below

According to one embodiment of the disclosure, an activation control 126, included on the stylet assembly 119, may be used to set the stylet 120 into a desired operating mode and selectively alter operability of the display 170 by the clinician to assist in medical device placement. For example, based on the modality of the stylet 120, the display 170 of the console 110 can be employed for optical modality-based guidance during catheter advancement through the body conduits or TLS modality to determine the physical state (e.g., length, form, shape, orientation, etc.) of the stylet 120. In one embodiment, information from multiple modes, such as optical, TLS or ECG for example, may be displayed concurrently (e.g., at least partially overlapping in time).

Referring still to FIG. 1A, the optical logic 180 is configured to support operability of the stylet assembly 119 and enable the return of information to the console 110, which may be used to determine the physical state associated with the stylet 120 along with monitored electrical signals such as ECG signaling via an electrical signaling logic 181 that supports receipt and processing of the received electrical signals from the stylet 120 (e.g., ports, analog-to-digital conversion logic, etc.). The physical state of the stylet 120 may be based on changes in characteristics of the reflected light signals 150 received at the console 110 from the stylet 120. The characteristics may include shifts in wavelength caused by strain on certain regions of the core fibers integrated within an optical fiber core 135 positioned within or operating as the stylet 120, as shown below. As discussed herein, the optical fiber core 135 may be comprised of core fibers 137₁-137_{M} (M=1 for a single core, and M≥2 for a multi-core), where the core fibers 137₁-137_{M} may collectively be referred to as core fiber(s) 137. Unless otherwise specified or the instant embodiment requires an alternative interpretation, embodiments discussed herein will refer to a multi-core optical fiber 135. From information associated with the reflected light signals 150, the console 110 may determine (through computation or extrapolation of the wavelength shifts) the physical state of the stylet 120, and that of the catheter 195 configured to receive the stylet 120.

According to one embodiment of the disclosure, as shown in FIG. 1A, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the multi-core optical fiber core 135 within the stylet 120. In one embodiment, the light source 182 is a tunable swept laser, although other suitable light sources can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the multi-core optical fiber 135 deployed within the stylet 120, and (ii) translate the reflected light signals 150 into reflection data (from repository 192), namely data in the form of electrical signals representative of the reflected light signals including wavelength shifts caused by strain. The reflected light signals 150 associated with different spectral widths may include reflected light signals 151 provided from sensors positioned in the center core fiber (reference) of the multi-core optical fiber 135 and reflected light signals 152 provided from sensors positioned in the periphery core fibers of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data (from repository 192) to the memory 165 for storage and processing by reflection data classification logic 190. The reflection data classification logic 190 may be configured to: (i) identify which core fibers pertain to which of the received reflection data (from repository 192) and (ii) segregate the reflection data stored with a repository 192 provided from reflected light signals 150 pertaining to similar regions of the stylet 120 or spectral widths into analysis groups. The reflection data for each analysis group is made available to shape sensing logic 194 for analytics.

According to one embodiment of the disclosure, the shape sensing logic 194 is configured to compare wavelength shifts measured by sensors deployed in each periphery core fiber at the same measurement region of the stylet 120 (or same spectral width) to the wavelength shift at a center core fiber of the multi-core optical fiber 135 positioned along central axis and operating as a neutral axis of bending. From these analytics, the shape sensing logic 194 may determine the shape the core fibers have taken in 3D space and may further determine the current physical state of the catheter 195 in 3D space for rendering on the display 170.

According to one embodiment of the disclosure, the shape sensing logic 194 may generate a rendering of the current physical state of the stylet 120 (and potentially the catheter 195), based on heuristics or run-time analytics. For example, the shape sensing logic 194 may be configured in accordance with machine-learning techniques to access a data store (library) with pre-stored data (e.g., images, etc.) pertaining to different regions of the stylet 120 (or catheter 195) in which reflected light from core fibers have previously experienced similar or identical wavelength shifts. From the pre-stored data, the current physical state of the stylet 120 (or catheter 195) may be rendered. Alternatively, as another example, the shape sensing logic 194 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the stylet 120 (and/or catheter 195), especially to enable guidance of the stylet 120, when positioned at a distal tip of the catheter 195, within the body conduits of the patient and at a desired destination within the body.

The console 110 may further include electrical signaling logic 181, which is positioned to receive one or more electrical signals from the stylet 120. The stylet 120 is configured to support both optical connectivity as well as electrical connectivity. The electrical signaling logic 181 receives the electrical signals (e.g., ECG signals) from the stylet 120 via the conductive medium. The electrical signals may be processed by electrical signal logic 196, executed by the processor 160, to determine ECG waveforms for display.

Referring to FIG. 1B, an alternative exemplary embodiment of a medical instrument monitoring system 100 is shown. Herein, the medical instrument monitoring system 100 features a console 110 and a medical instrument 130 communicatively coupled to the console 110. For this embodiment, the medical instrument 130 corresponds to a catheter, which features an integrated tubing with two or more lumen extending between a proximal end 131 and a distal end 132 of the integrated tubing. The integrated tubing (sometimes referred to as “catheter tubing”) is in communication with one or more extension legs 140 via a bifurcation hub 142. An optical-based catheter connector 144 may be included on a proximal end of at least one of the extension legs 140 to enable the catheter 130 to operably connect to the console 110 via an interconnect 145 or another suitable component. Herein, the interconnect 145 may include a connector 146 that, when coupled to the optical-based catheter connector 144, establishes optical connectivity between one or more optical fibers 147 (hereinafter, “optical fiber(s)”) included as part of the interconnect 145 and core fibers 137 deployed within the catheter 130 and integrated into the tubing. Alternatively, a different combination of connectors, including one or more adapters, may be used to optically connect the optical fiber(s) 147 to the core fibers 137 within the catheter 130. The core fibers 137 deployed within the catheter 130 as illustrated in FIG. 1B include the same characteristics and perform the same functionalities as the core fibers 137 deployed within the stylet 120 of FIG. 1A.

The optical logic 180 is configured to support graphical rendering of the catheter 130, most notably the integrated tubing of the catheter 130, based on characteristics of the reflected light signals 150 received from the catheter 130. The characteristics may include shifts in wavelength caused by strain on certain regions of the core fibers 137 integrated within (or along) a wall of the integrated tubing, which may be used to determine (through computation or extrapolation of the wavelength shifts) the physical state of the catheter 130, notably its integrated tubing or a portion of the integrated tubing such as a tip or distal end of the tubing to read fluctuations (real-time movement) of the tip (or distal end).

More specifically, the optical logic 180 includes a light source 182. The light source 182 is configured to transmit the broadband incident light 155 for propagation over the optical fiber(s) 147 included in the interconnect 145, which are optically connected to multiple core fibers 137 within the catheter tubing. Herein, the optical receiver 184 is configured to: (i) receive returned optical signals, namely reflected light signals 150 received from optical fiber-based reflective gratings (sensors) fabricated within each of the core fibers 137 deployed within the catheter 130, and (ii) translate the reflected light signals 150 into reflection data (from repository 192), namely data in the form of electrical signals representative of the reflected light signals including wavelength shifts caused by strain. The reflected light signals 150 associated with different spectral widths include reflected light signals 151 provided from sensors positioned in the center core fiber (reference) of the catheter 130 and reflected light signals 152 provided from sensors positioned in the outer core fibers of the catheter 130, as described below.

As noted above, the shape sensing logic 194 is configured to compare wavelength shifts measured by sensors deployed in each outer core fiber at the same measurement region of the catheter (or same spectral width) to the wavelength shift at the center core fiber positioned along central axis and operating as a neutral axis of bending. From these analytics, the shape sensing logic 190 may determine the shape the core fibers have taken in 3D space and may further determine the current physical state of the catheter 130 in 3D space for rendering on the display 170.

According to one embodiment of the disclosure, the shape sensing logic 194 may generate a rendering of the current physical state of the catheter 130, especially the integrated tubing, based on heuristics or run-time analytics. For example, the shape sensing logic 194 may be configured in accordance with machine-learning techniques to access a data store (library) with pre-stored data (e.g., images, etc.) pertaining to different regions of the catheter 130 in which the core fibers 137 experienced similar or identical wavelength shifts. From the pre-stored data, the current physical state of the catheter 130 may be rendered. Alternatively, as another example, the shape sensing logic 194 may be configured to determine, during run-time, changes in the physical state of each region of the catheter 130, notably the tubing, based on at least (i) resultant wavelength shifts experienced by the core fibers 137 and (ii) the relationship of these wavelength shifts generated by sensors positioned along different outer core fibers at the same cross-sectional region of the catheter 130 to the wavelength shift generated by a sensor of the center core fiber at the same cross-sectional region. It is contemplated that other processes and procedures may be performed to utilize the wavelength shifts as measured by sensors along each of the core fibers 137 to render appropriate changes in the physical state of the catheter 130.

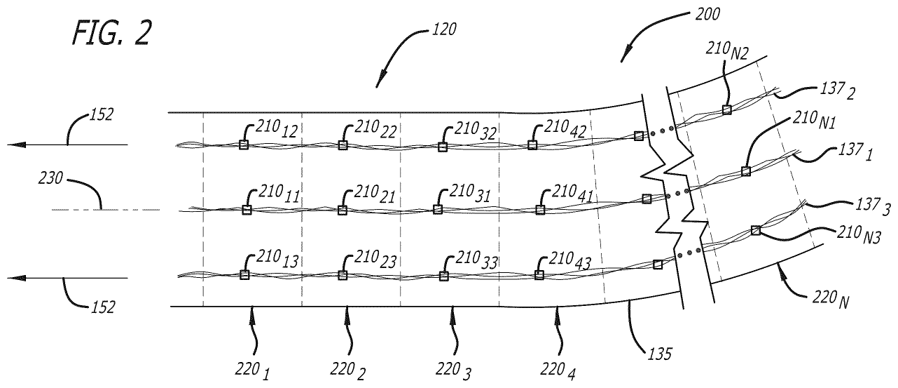

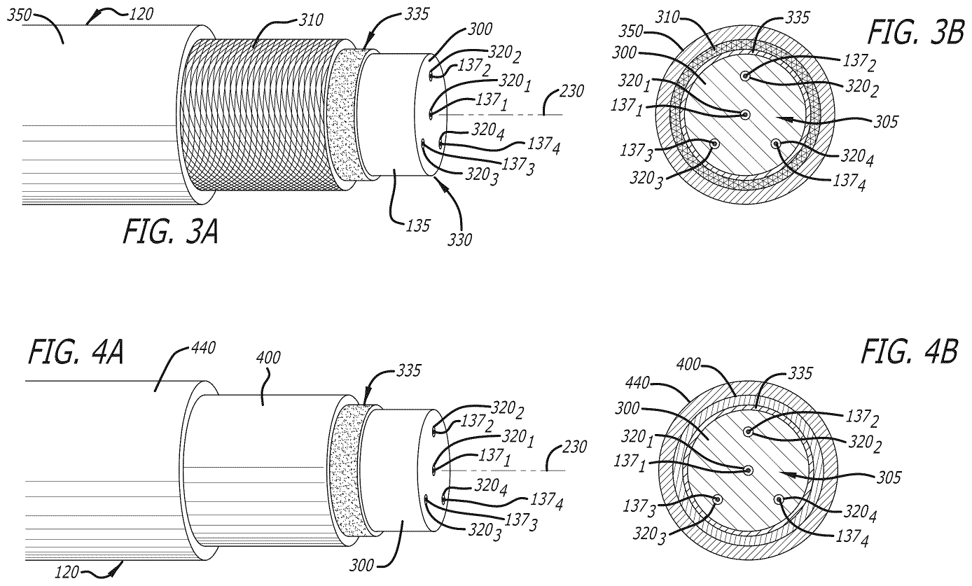

Referring to FIG. 2, an exemplary embodiment of a structure of a section of the multi-core optical fiber included within the stylet 120 of FIG. 1A is shown in accordance with some embodiments. The multi-core optical fiber section 200 of the multi-core optical fiber 135 depicts certain core fibers 137₁-137_{M} (M≥2, M=4 as shown, see FIG. 3A) along with the spatial relationship between sensors (e.g., reflective gratings) 210₁₁-210_{NM} (N≥2; M≥2) present within the core fibers 137₁-137_{M}, respectively. As noted above, the core fibers 137₁-137_{M} may be collectively referred to as “the core fibers 137.”

As shown, the section 200 is subdivided into a plurality of cross-sectional regions 220₁-220_{N}, where each cross-sectional region 220₁-220_{N} corresponds to reflective gratings 210₁₁-210₁₄ . . . 210_{N1}-210_{N4}. Some or all of the cross-sectional regions 220₁ . . . 220_{N} may be static (e.g., prescribed length) or may be dynamic (e.g., vary in size among the regions 220₁ . . . 220_{N}). A first core fiber 137₁ is positioned substantially along a center (neutral) axis 230 while core fiber 137₂ may be oriented within the cladding of the multi-core optical fiber 135, from a cross-sectional, front-facing perspective, to be position on “top” the first core fiber 137₁. In this deployment, the core fibers 137₃ and 137₄ may be positioned “bottom left” and “bottom right” of the first core fiber 137₁. As examples, FIGS. 3A-4B provides illustrations of such.

Referencing the first core fiber 137₁ as an illustrative example, when the stylet 120 is operative, each of the reflective gratings 210₁-210_{N} reflects light for a different spectral width. As shown, each of the gratings 210ᵢᵢ-210_{Ni} (1≤i≤M) is associated with a different, specific spectral width, which would be represented by different center frequencies of f₁ . . . f_{N}, where neighboring spectral widths reflected by neighboring gratings are non-overlapping according to one embodiment of the disclosure.

Herein, positioned in different core fibers 137₂-137₃ but along at the same cross-sectional regions 220-220_{N} of the multi-core optical fiber 135, the gratings 210₁₂-210_{N2} and 210₁₃-210_{N3} are configured to reflect incoming light at same (or substantially similar) center frequency. As a result, the reflected light returns information that allows for a determination of the physical state of the optical fibers 137 (and the stylet 120) based on wavelength shifts measured from the returned, reflected light. In particular, strain (e.g., compression or tension) applied to the multi-core optical fiber 135 (e.g., at least core fibers 137₂-137₃) results in wavelength shifts associated with the returned, reflected light. Based on different locations, the core fibers 137₁-137₄ experience different types and degree of strain based on angular path changes as the stylet 120 advances in the patient.

For example, with respect to the multi-core optical fiber section 200 of FIG. 2, in response to angular (e.g., radial) movement of the stylet 120 is in the left-veering direction, the fourth core fiber 137₄ (see FIG. 3A) of the multi-core optical fiber 135 with the shortest radius during movement (e.g., core fiber closest to a direction of angular change) would exhibit compression (e.g., forces to shorten length). At the same time, the third core fiber 137₃ with the longest radius during movement (e.g., core fiber furthest from the direction of angular change) would exhibit tension (e.g., forces to increase length). As these forces are different and unequal, the reflected light from reflective gratings 210_{N2} and 210_{N3} associated with the core fibers 137₂ and 137₃ will exhibit different changes in wavelength. The differences in wavelength shift of the reflected light signals 150 can be used to extrapolate the physical configuration of the stylet 120 by determining the degrees of wavelength change caused by compression/tension for each of the periphery fibers (e.g., the second core fiber 137₂ and the third core fiber 137₃) in comparison to the wavelength of the reference core fiber (e.g., first core fiber 137₁) located along the neutral axis 230 of the multi-core optical fiber 135. These degrees of wavelength change may be used to extrapolate the physical state of the stylet 120. The reflected light signals 150 are reflected back to the console 110 via individual paths over a particular core fiber 137₁-137_{M}.

Referring to FIG. 3A, a first exemplary embodiment of the stylet of FIG. 1A supporting both an optical and electrical signaling is shown in accordance with some embodiments. Herein, the stylet 120 features a centrally located multi-core optical fiber 135, which includes a cladding 300 and a plurality of core fibers 137₁-137_{M} (M≥2; M=4) residing within a corresponding plurality of lumens 320₁-320_{M}. While the multi-core optical fiber 135 is illustrated within four (4) core fibers 137₁-137₄, a greater number of core fibers 137₁-137_{M} (M>4) may be deployed to provide a more detailed three-dimensional sensing of the physical state (e.g., shape, etc.) of the multi-core optical fiber 135 and the stylet 120 deploying the optical fiber 135.

For this embodiment of the disclosure, the multi-core optical fiber 135 is encapsulated within a concentric braided tubing 310 positioned over a low coefficient of friction layer 335. The braided tubing 310 may feature a “mesh” construction, in which the spacing between the intersecting conductive elements is selected based on the degree of rigidity desired for the stylet 120, as a greater spacing may provide a lesser rigidity, and thereby, a more pliable stylet 120.

According to this embodiment of the disclosure, as shown in FIGS. 3A-3B, the core fibers 137₁-137₄ include (i) a central core fiber 137₁ and (ii) a plurality of periphery core fibers 137₂-137₄, which are maintained within lumens 320₁-320₄ formed in the cladding 300. According to one embodiment of the disclosure, one or more of the lumens 320₁-320₄ may be configured with a diameter sized to be greater than the diameter of the core fibers 137₁-137₄. By avoiding a majority of the surface area of the core fibers 137₁-137₄ from being in direct physical contact with a wall surface of the lumens 320₁-320₄, the wavelength changes to the incident light are caused by angular deviations in the multi-core optical fiber 135 thereby reducing influence of compression and tension forces being applied to the walls of the lumens 320₁-320_{M}, not the core fibers 137₁-137_{M} themselves.

As further shown in FIGS. 3A-3B, the core fibers 137₁-137₄ may include central core fiber 137₁ residing within a first lumen 320₁ formed along the first neutral axis 230 and a plurality of core fibers 137₂-137₄ residing within lumens 320₂-320₄ each formed within different areas of the cladding 300 radiating from the first neutral axis 230. In general, the core fibers 137₂-137₄, exclusive of the central core fiber 137₁, may be positioned at different areas within a cross-sectional area 305 of the cladding 300 to provide sufficient separation to enable three-dimensional sensing of the multi-core optical fiber 135 based on changes in wavelength of incident light propagating through the core fibers 137₂-137₄ and reflected back to the console for analysis.

For example, where the cladding 300 features a circular cross-sectional area 305 as shown in FIG. 3B, the core fibers 137₂-137₄ may be positioned substantially equidistant from each other as measured along a perimeter of the cladding 300, such as at “top” (12 o'clock), “bottom-left” (8 o'clock) and “bottom-right” (4 o'clock) locations as shown. Hence, in general terms, the core fibers 137₂-137₄ may be positioned within different segments of the cross-sectional area 305. Where the cross-sectional area 305 of the cladding 300 has a distal tip 330 and features a polygon cross-sectional shape (e.g., triangular, square, rectangular, pentagon, hexagon, octagon, etc.), the central core fiber 137₁ may be located at or near a center of the polygon shape, while the remaining core fibers 137₂-137_{M} may be located proximate to angles between intersecting sides of the polygon shape.

Referring still to FIGS. 3A-3B, operating as the conductive medium for the stylet 120, the braided tubing 310 provides mechanical integrity to the multi-core optical fiber 135 and operates as a conductive pathway for electrical signals. For example, the braided tubing 310 may be exposed to a distal tip of the stylet 120. The cladding 300 and the braided tubing 310, which is positioned concentrically surrounding a circumference of the cladding 300, are contained within the same insulating layer 350. The insulating layer 350 may be a sheath or conduit made of protective, insulating (e.g., non-conductive) material that encapsulates both for the cladding 300 and the braided tubing 310, as shown.

Referring to FIG. 4A, a second exemplary embodiment of the stylet of FIG. 1A is shown in accordance with some embodiments. Herein, the stylet 120 features the multi-core optical fiber 135 described above and shown in FIG. 3A, which includes the cladding 300 and the first plurality of core fibers 137₁-137_{M} (M≥3; M=4 for embodiment) residing within the corresponding plurality of lumens 320₁-320_{M}. For this embodiment of the disclosure, the multi-core optical fiber 135 includes the central core fiber 137₁ residing within the first lumen 320₁ formed along the first neutral axis 230 and the second plurality of core fibers 137₂-137₄ residing within corresponding lumens 320₂-320₄ positioned in different segments within the cross-sectional area 305 of the cladding 300. Herein, the multi-core optical fiber 135 is encapsulated within a conductive tubing 400. The conductive tubing 400 may feature a “hollow” conductive cylindrical member concentrically encapsulating the multi-core optical fiber 135.

Referring to FIGS. 4A-4B, operating as a conductive medium for the stylet 120 in the transfer of electrical signals (e.g., ECG signals) to the console, the conductive tubing 400 may be exposed up to a tip 410 of the stylet 120. For this embodiment of the disclosure, a conductive epoxy 420 (e.g., metal-based epoxy such as a silver epoxy) may be affixed to the tip 410 and similarly joined with a termination/connection point created at a proximal end 430 of the stylet 120. The cladding 300 and the conductive tubing 400, which is positioned concentrically surrounding a circumference of the cladding 300, are contained within the same insulating layer 440. The insulating layer 440 may be a protective conduit encapsulating both for the cladding 300 and the conductive tubing 400, as shown.

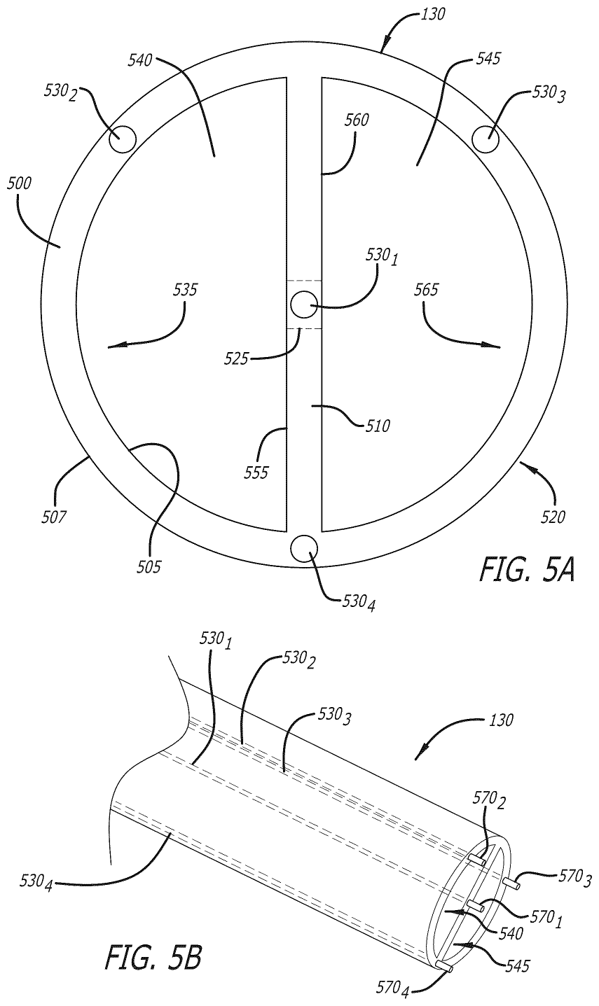

Referring to FIG. 5A, an elevation view of a first illustrative embodiment of a catheter including integrated tubing, a diametrically disposed septum, and micro-lumens formed within the tubing and septum is shown in accordance with some embodiments. Herein, the catheter 130 includes integrated tubing, the diametrically disposed septum 510, and the plurality of micro-lumens 530₁-530₄ which, for this embodiment, are fabricated to reside within the wall 500 of the integrated tubing of the catheter 130 and within the septum 510. In particular, the septum 510 separates a single lumen, formed by the inner surface 505 of the wall 500 of the catheter 130, into multiple lumens, namely two lumens 540 and 545 as shown. Herein, the first lumen 540 is formed between a first arc-shaped portion 535 of the inner surface 505 of the wall 500 forming the catheter 130 and a first outer surface 555 of the septum 510 extending longitudinally within the catheter 130. The second lumen 545 is formed between a second arc-shaped portion 565 of the inner surface 505 of the wall 500 forming the catheter 130 and a second outer surfaces 560 of the septum 510.

According to one embodiment of the disclosure, the two lumens 540 and 545 have approximately the same volume. However, the septum 510 need not separate the tubing into two equal lumens. For example, instead of the septum 510 extending vertically (12 o'clock to 6 o'clock) from a front-facing, cross-sectional perspective of the tubing, the septum 510 could extend horizontally (3 o'clock to 9 o'clock), diagonally (1 o'clock to 7 o'clock; 10 o'clock to 4 o'clock) or angularly (2 o'clock to 10 o'clock). In the later configuration, each of the lumens 540 and 545 of the catheter 130 would have a different volume.

With respect to the plurality of micro-lumens 530₁-530₄, the first micro-lumen 530₁ is fabricated within the septum 510 at or near the cross-sectional center 525 of the integrated tubing. For this embodiment, three micro-lumens 530₂-530₄ are fabricated to reside within the wall 500 of the catheter 130. In particular, a second micro-lumen 530₂ is fabricated within the wall 500 of the catheter 130, namely between the inner surface 505 and outer surface 507 of the first arc-shaped portion 535 of the wall 500. Similarly, the third micro-lumen 5303 is also fabricated within the wall 500 of the catheter 130, namely between the inner and outer surfaces 505/507 of the second arc-shaped portion 555 of the wall 500. The fourth micro-lumen 530₄ is also fabricated within the inner and outer surfaces 505/507 of the wall 500 that are aligned with the septum 510.

According to one embodiment of the disclosure, as shown in FIG. 5A, the micro-lumens 530₂-530₄ are positioned in accordance with a “top-left” (10 o'clock), “top-right” (2 o'clock) and “bottom” (6 o'clock) layout from a front-facing, cross-sectional perspective. Of course, the micro-lumens 530₂-530₄ may be positioned differently, provided that the micro-lumens 530₂-530₄ are spatially separated along the circumference 520 of the catheter 130 to ensure a more robust collection of reflected light signals from the outer core fibers 570₂-570₄ when installed. For example, two or more of micro-lumens (e.g., micro-lumens 530₂ and 530₄) may be positioned at different quadrants along the circumference 520 of the catheter wall 500.

Referring to FIG. 5B, a perspective view of the first illustrative embodiment of the catheter of FIG. 5A including core fibers installed within the micro-lumens is shown in accordance with some embodiments. According to one embodiment of the disclosure, the second plurality of micro-lumens 530₂-530₄ are sized to retain corresponding outer core fibers 570₂-570₄, where the diameter of each of the second plurality of micro-lumens 530₂-530₄ may be sized just larger than the diameters of the outer core fibers 570₂-570₄. The size differences between a diameter of a single core fiber and a diameter of any of the micro-lumen 530₁-530₄ may range between 0.001 micrometers (μm) and 1000 μm, for example. As a result, the cross-sectional areas of the outer core fibers 570₂-570₄ would be less than the cross-sectional areas of the corresponding micro-lumens 530₂-530₄. A “larger” micro-lumen (e.g., micro-lumen 530₂) may better isolate external strain being applied to the outer core fiber 570₂ from strain directly applied to the catheter 130 itself. Similarly, the first micro-lumen 530₁ may be sized to retain the center core fiber 570₁, where the diameter of the first micro-lumen 530₁ may be sized just larger than the diameter of the center core fiber 570₁.

As an alternative embodiment of the disclosure, one or more of the micro-lumens 530₁-530₄ may be sized with a diameter that exceeds the diameter of the corresponding one or more core fibers 570₁-570₄. However, at least one of the micro-lumens 530₁-530₄ is sized to fixedly retain their corresponding core fiber (e.g., core fiber retained with no spacing between its lateral surface and the interior wall surface of its corresponding micro-lumen). As yet another alternative embodiment of the disclosure, all the micro-lumens 530₁-530₄ are sized with a diameter to fixedly retain the core fibers 570₁-570₄.

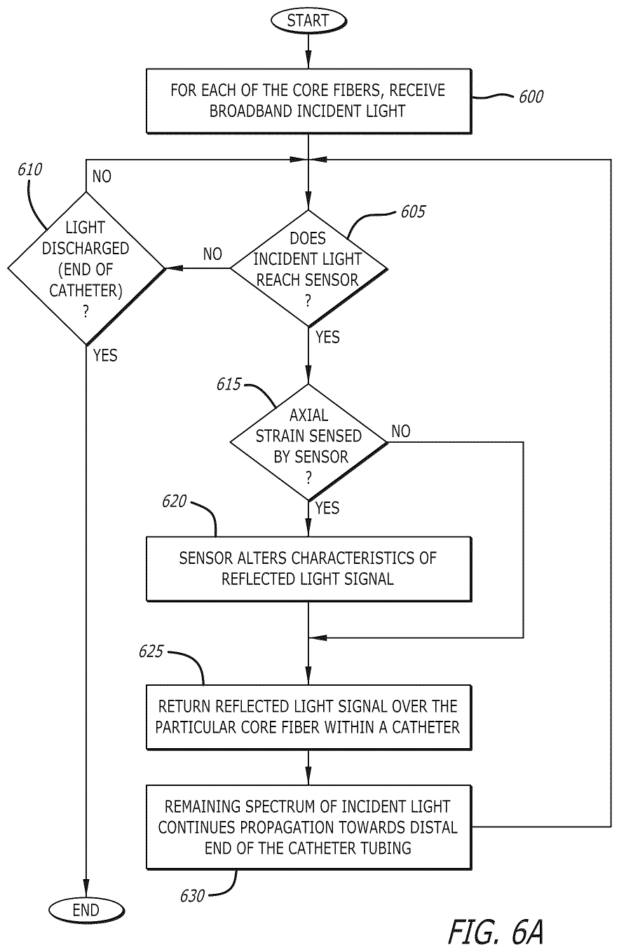

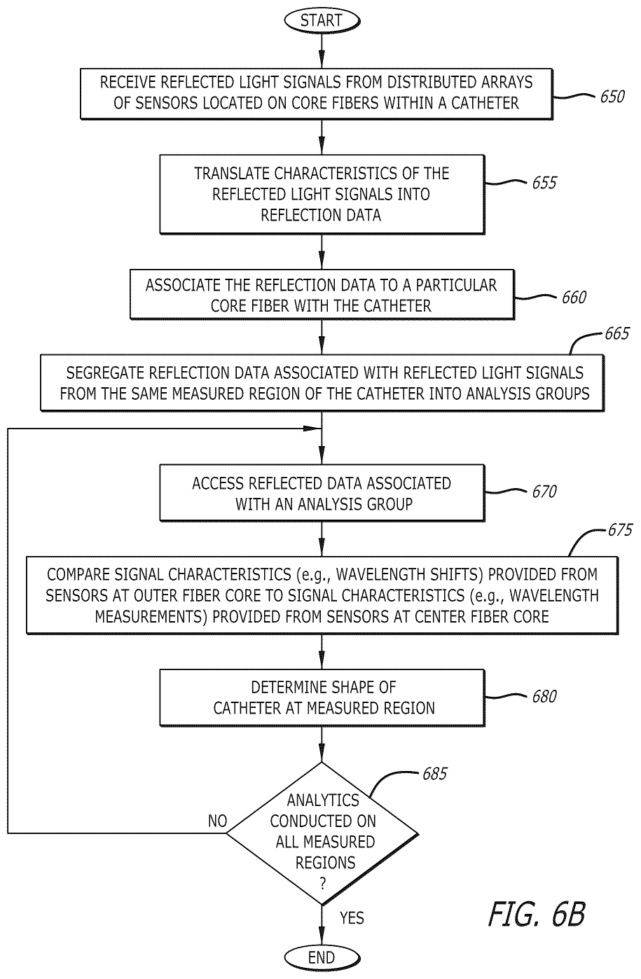

Referring to FIGS. 6A-6B, flowcharts of methods of operations conducted by the medical instrument monitoring system of FIGS. 1A-1B to achieve optic 3D shape sensing are shown in accordance with some embodiments. Herein, the catheter includes at least one septum spanning across a diameter of the tubing wall and continuing longitudinally to subdivide the tubing wall. The medial portion of the septum is fabricated with a first micro-lumen, where the first micro-lumen is coaxial with the central axis of the catheter tubing. The first micro-lumen is configured to retain a center core fiber. Two or more micro-lumen, other than the first micro-lumen, are positioned at different locations circumferentially spaced along the wall of the catheter tubing. For example, two or more of the second plurality of micro-lumens may be positioned at different quadrants along the circumference of the catheter wall.

Furthermore, each core fiber includes a plurality of sensors spatially distributed along its length between at least the proximal and distal ends of the catheter tubing. This array of sensors is distributed to position sensors at different regions of the core fiber to enable distributed measurements of strain throughout the entire length or a selected portion of the catheter tubing. These distributed measurements may be conveyed through reflected light of different spectral widths (e.g., specific wavelength or specific wavelength ranges) that undergoes certain wavelength shifts based on the type and degree of strain.

According to one embodiment of the disclosure, as shown in FIG. 6A, for each core fiber, broadband incident light is supplied to propagate through a particular core fiber (block 600). Unless discharged, upon the incident light reaching a sensor of a distributed array of sensors measuring strain on a particular core fiber, light of a prescribed spectral width associated with the first sensor is to be reflected back to an optical receiver within a console (blocks 605-610). Herein, the sensor alters characteristics of the reflected light signal to identify the type and degree of strain on the particular core fiber as measured by the first sensor (blocks 615-620). According to one embodiment of the disclosure, the alteration in characteristics of the reflected light signal may signify a change (shift) in the wavelength of the reflected light signal from the wavelength of the incident light signal associated with the prescribed spectral width. The sensor returns the reflected light signal over the core fiber and the remaining spectrum of the incident light continues propagation through the core fiber toward a distal end of the catheter tubing (blocks 625-630). The remaining spectrum of the incident light may encounter other sensors of the distributed array of sensors, where each of these sensors would operate as set forth in blocks 605-630 until the last sensor of the distributed array of sensors returns the reflected light signal associated with its assigned spectral width and the remaining spectrum is discharged as illumination.

Referring now to FIG. 6B, during operation, multiple reflected light signals are returned to the console from each of the plurality of core fibers residing within the corresponding plurality of micro-lumens formed within a catheter, such as the catheter of FIG. 1B. In particular, the optical receiver receives reflected light signals from the distributed arrays of sensors located on the center core fiber and the outer core fibers and translates the reflected light signals into reflection data, namely electrical signals representative of the reflected light signals including wavelength shifts caused by strain (blocks 650-655). The reflection data classification logic is configured to identify which core fibers pertain to which reflection data and segregate reflection data provided from reflected light signals pertaining to a particular measurement region (or similar spectral width) into analysis groups (block 660-665).

Each analysis group of reflection data is provided to shape sensing logic for analytics (block 670). Herein, the shape sensing logic compares wavelength shifts at each outer core fiber with the wavelength shift at the center core fiber positioned along central axis and operating as a neutral axis of bending (block 675). From these analytics, on all analytic groups (e.g., reflected light signals from sensors in all or most of the core fibers), the shape sensing logic may determine the shape the core fibers have taken in three-dimensional space, from which the shape sensing logic can determine the current physical state of the catheter in three-dimension space (blocks 680-685).

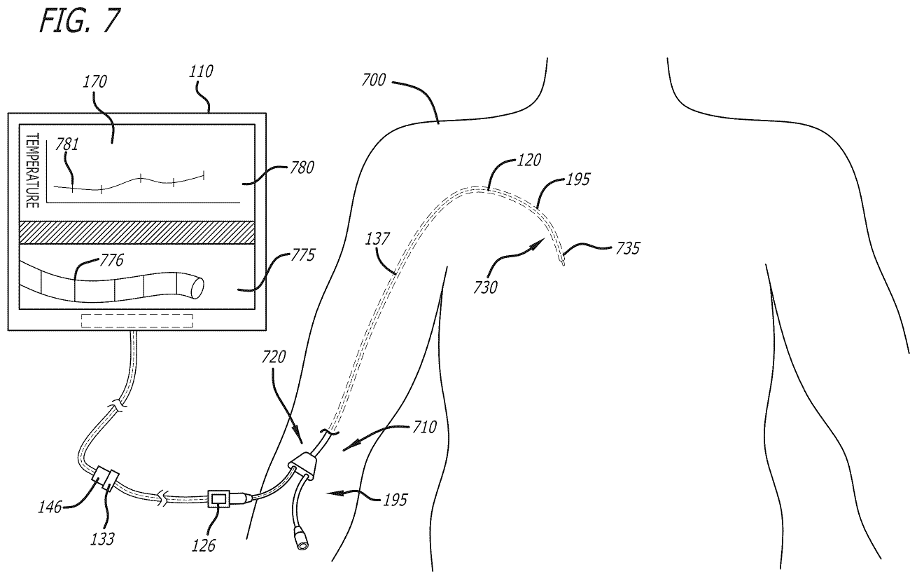

Referring to FIG. 7, an exemplary embodiment of the medical instrument monitoring system of FIG. 1A during operation and insertion of the catheter into a patient are shown in accordance with some embodiments. Herein, the catheter 195 generally includes integrated tubing with a proximal portion 720 that generally remains exterior to the patient 700 and a distal portion 730 that generally resides within the patient vasculature after placement is complete, where the catheter 195 enters the vasculature at insertion site 710. The stylet 120 may be advanced through the catheter 195 to a desired position within the patient vasculature such that a distal end (or tip) 735 of the stylet 120 (and hence a distal end of the catheter 195) is proximate the patient's heart, such as in the lower one-third (⅓) portion of the Superior Vena Cava (“SVC”) for example. For this embodiment, various instruments may be placed at the distal end 735 of the stylet 120 and/or the catheter 195 to measure pressure of blood in a certain heart chamber and in the blood vessels, view an interior of blood vessels, or the like.

The console connector 133 enables the stylet 120 to be operably connected to the console 110 via the interconnect 145 (FIG. 1A). Herein, the connector 146 is configured to engage (mate) with the console connector 133 to allow for the propagation of light between the console 110 and the stylet assembly 119 (particularly the stylet 120) as well as the propagation of electrical signals from the stylet 120 to the console 110.

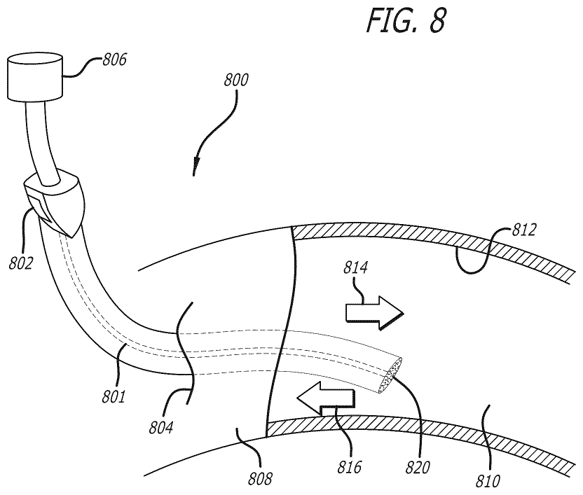

Referring now to FIG. 8, a cross sectional perspective view of a body conduit having a medical instrument advancing therein where the medical instrument includes an optical fiber having a single sensor disposed at a distal tip shown to be receiving and reflecting light is shown in accordance with some embodiments. The cross-sectional perspective view of the body conduit 812 illustrates an interior 810 with a specific temperature. Additionally, FIG. 8 illustrates a medical instrument 806 (e.g., a catheter or stylet) having been inserted through a skin 808 of a patient at an entry site 804 and advancing through the body conduit 812. The medical instrument 806 includes an optical fiber 801 having one or more core fibers, with each core fiber configured to receive incident light signal 814 from a console (e.g., the light source 182 of the console 110) such that the incident light signal 814 propagates to a distal end of the core fiber.

In some embodiments, the optical fiber 801 may include a reference sensor 802 positioned externally to the body of a patient. The reference sensor 802 is positioned at a location with a known temperature (e.g., ambient or room temperature) and to reflect light of a specific spectral width back to the console 110 for processing. In some embodiments, a thermometer may be positioned adjacent the reference sensor 802 enabling for detection of the known temperature. As shown, the incident light signal 814 has propagated past the reference sensor 802, through the length of the optical fiber 801, until reaching a distal sensor 820 disposed at or adjacent the distal end of the core fiber. The distal sensor receives incident light signal 814 and is configured to reflect light of a specific spectral width (e.g., reflected light single 816). The reflected lighted signal 816 propagates the length of the optical fiber 801 back to the console 110 for processing. Specifically, the temperature sensing logic 198 may determine an unknown temperature at the distal tip of the medical instrument 806 by comparing the reflected light data transmitted by the distal sensor 820 with the reflected light data transmitted by the reference sensor 802, which is associated with the known temperature.

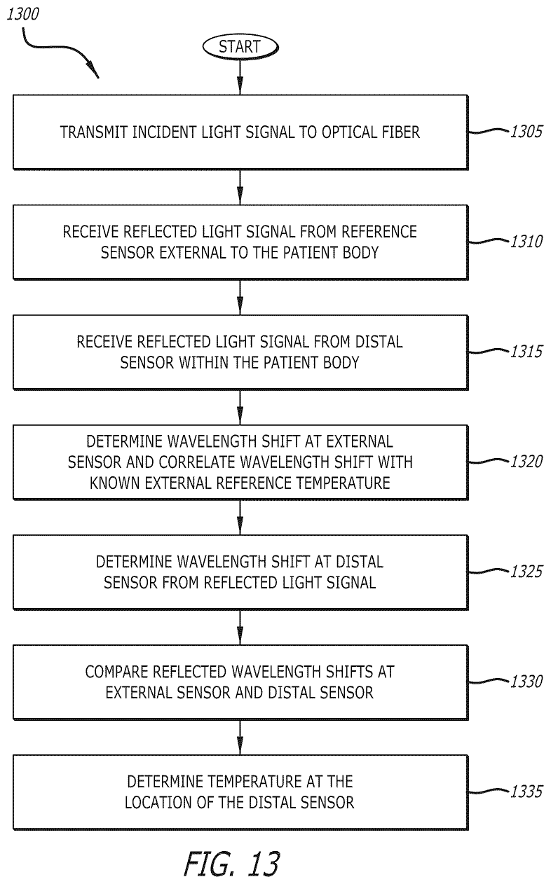

More specifically, the reflected light signal 816 is received by the optical receiver 184, which is configured to: (i) receive returned optical signals (e.g., the reflected light signal 816), and (ii) translate the reflected light signal 816 into reflection data, namely data in the form of electrical signals representative of the reflected light signal 816. In some embodiments, the reflection data may comprise various parameters, including a reflected wavelength, a reflected wavelength shift, a sensor location on the optical fiber 801, etc. The temperature sensing logic 198 subsequently analyzes the reflection data to determine a temperature within the body conduit 812 at the distal end of the optical fiber 801, as discussed below and illustrated with respect to at least FIG. 13. More specifically, the temperature within the body conduit 812 may alter characteristics of the distal sensor thereby altering the reflected light signal 816. For example, the temperature (e.g., the ambient temperature of the body conduit 812) may (i) change the refractive index of the distal sensor 820 (e.g., higher temperatures increase a refractive index of the sensor, thereby increasing the wavelength of the reflected light signal) and/or (ii) change a physical dimension of the optical fiber 801 (e.g., higher temperatures will increase a thermal expansion of the optical fiber, thereby increasing a refection period of the distal sensor, thereby increasing the wavelength of the reflected light signal). Stated differently, variations in temperature at the location proximate the distal sensor 820 may cause the reflected light signal 816 to undergo a wavelength shift. In some instances, the relationship between temperature and wavelength shift may be described as:

$$Δ {λ}_{B} = {λ}_{B} ( α + ξ ) Δ T$$

which becomes:

$$T = {T}_{R e f} + \frac{Δ {λ}_{B}}{{λ}_{B} ( α + ξ )}$$

where:
T is defined as the temperature at the sensing point
T_{Ref} is defined as the reference temperature
T is defined as the change in temperature
λ_{B} is defined as the initial wavelength (wavelength at which sensor reflects light)
Δλ_{B} is defined as the wavelength shift of the reflected light signal
ξ is defined as the thermo-optic coefficient
α is defined as the coefficient of thermal expansion
   It should be appreciated that the thermo-optic coefficient and the coefficient of thermal expansion can be empirically determined and are dependent on the material of the fiber optic core.

In certain embodiments, the temperature sensing logic 198 of the console 110 is configured to analyze the reflected light signal 816 to determine a temperature of the body of the patient at a sensing point (e.g., where the distal sensor 820 is located with respect to the body of the patient). Specifically, temperature changes at the sensing point alter characteristics of the distal sensor 820, causing the distal sensor to reflect light with a wavelength that may be shifted to a higher or a lower value. The corresponding wavelength shifts are obtained by the console 101 through analysis of the reflected light signal 816. The console 110 correlates the observed wavelength shift to the determine the temperature within the body of the patient at the sensing point, as discussed below with respect to at least FIG. 13.

In certain embodiments, the temperature, once determined, may be utilized in determining a location of the distal tip of the optical fiber 801 within the body conduit 812. For example, certain temperatures may correspond to particular locations within the patient body such that, based on an insertion site and, optionally other location or navigation data (e.g., the shape sensing functionality discussed above, ECG, etc.), a location of the distal tip of the optical fiber 801 may be determined or at least approximated to be within a certain region of the patient body. For example, the determined temperature may provide an indication that the distal tip of the optical fiber 801 (and hence the distal tip of the medical instrument 806) has deviated from its intended path of advancement since temperature varies at different locations throughout the patient body (e.g., blood in peripheral regions, like limbs or other extremities, may be cooler than blood flowing closer to the heart, particularly the aorta). If the temperature sensing logic 198 detects insertion past a sensing point, the medical instrument monitoring system may optionally provide an alert indicating that the medical instrument 806 has traversed past the sensing point.

Additionally in some embodiments, the determined temperature level may be utilized in verifying whether a distal tip has entered an area having a variant or abnormal temperature. For example, areas with inflammation or infection generate more heat, while areas with reduced circulation (e.g., a blocked blood vessel), generate less heat. Specifically, the console 110 may compare the detected temperature to a known or expected temperature at that specific location. If the detected temperature differs from the known or expected temperature by a predetermined threshold, the system may identify abnormalities and/or provide data (e.g., a location) on the variant temperature source. In addition, in some embodiments, the location of the distal tip may be determined as being within a vein or an artery. Veins and arteries may have distinct temperatures; thus, the temperature sensing logic 198 may compare the determined temperature to known or expected temperature of each of a vein and an artery to determine which vessel the determined temperature most closely corresponds.

In certain embodiments, the determined temperature level may be utilized in detecting aspiration within a patient. As used herein, the term “aspiration” generally refers to an inhalation of foreign materials, such as food, liquid, or gastric contents, into the airways. It should be understood that the core body temperature of a patient diagnosed with aspiration or aspiration pneumonia may increase. Therefore, the temperature sensing logic 198 may compare the determined temperature to a known or expected temperature. If the determined temperature exceeds a threshold value, the console 110 based on this data, or in combination with other metrics, may detect or indicate aspiration within a patient.

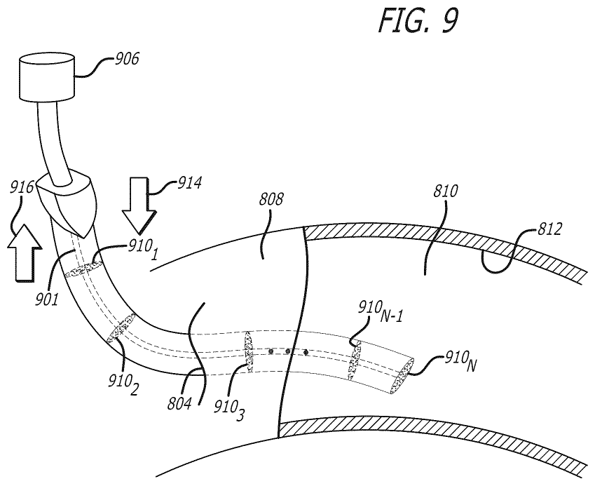

Referring to FIG. 9, a cross sectional perspective view of the blood vessel of FIG. 8 having the medical instrument advancing therein where the medical instrument includes an optical fiber having plurality of sensors distributed throughout a length of the fiber, wherein each sensor is shown to be receiving and reflecting light is shown in accordance with some embodiments. The cross-sectional perspective view of the body conduit 812 illustrates an interior 810 with a specific temperature. Additionally, FIG. 9 illustrates a medical instrument 806 (e.g., a catheter or a stylet) inserted through a skin 808 of a patient at an entry site 804 and advancing through the body conduit 812. The medical instrument 806 includes an optical fiber 801 having one or more core fibers, with each core fiber configured to receive incident light signal 914 from a console (e.g., the light source 182 of the console 110) such that the incident light signal 914 propagates to a distal end of the core fiber.

Furthermore, each core fiber includes a plurality of sensors (e.g., sensors 910₁-910_{N}) spatially distributed along the length of the core fiber between at least the proximal and distal ends of the medical instrument 806. The plurality of sensors 910 are positioned along the core fiber to enable distributed measurements of temperature throughout the entire length or a selected portion of the medical instrument 806. These distributed measurements may be conveyed through reflected light of different spectral widths (e.g., specific wavelength or specific wavelength ranges) that undergo certain wavelength shifts based, in part, on the ambient temperature of each of the plurality of sensors 910.

According to one embodiment of the disclosure, for each core fiber, an incident light signal 914 is supplied to propagate through a particular core fiber. Unless discharged, upon the incident light reaching a first sensor 910₁ of a distributed array of sensors 910, light of a prescribed spectral width associated with the first sensor 910₁ is to be reflected back to an optical receiver within a console. Herein, the sensor 910₁ may be altered by temperature changes at the sensing point, causing the sensor 910₁ to reflect a light signal having a shifted wavelength. The remaining spectrum of the incident light may encounter other sensors of the distributed array of sensors 910 at corresponding sensing points, where each of these sensors (e.g., 910₁, 910₂, 910₃, and 910_{N-1}) may alter characteristics of the incident light signal 914 and return reflected light signal 916 until a last sensor 910_{N} of the distributed array of sensors 910 returns the reflected light signal 916 associated with its assigned spectral width and the remaining spectrum is discharged as illumination. The reflected light signal 916 is analyzed by the temperature sensing logic 198 to determine a relative or absolute temperature at each of the sensors 910.

In some embodiments, once the temperature at each senor has been determined, the temperature sensing logic 198 may be configured to generate a temperature gradient 780. Specifically, the temperature sensing logic 198 may correlate a recorded temperature value with a corresponding sensor location. The console 110 may then graphically display the temperature value at a sensor position within the medical instrument and/or within the body of the patient. For example, the temperature value 781 corresponds to the sensor 776. In some embodiments, as is shown in FIG. 7, the graphical display may be configured to vertically align a temperature value with the corresponding sensor. In such instances, the graphical display may automatically change the scale of the displays of the temperature gradient 780 and a physical rendering 775 of the medical instrument in order to accommodate additional sensors and temperature readings as the medical instrument advances through the patient body. Further, the temperature gradient 780 may update at set intervals, such as every second, every 5 seconds, etc. In some embodiments, the temperature gradient 780 may be updated as temperature readings are determined from each sensor, which may be understood as updating in “real-time.” Based on the positioning of the plurality of sensors 920, this configuration enables the system to map temperature variations across different sections of the medical instrument. It should be appreciated that the temperature sensing logic 198 can produce a graphical profile of temperature changes throughout the medical instrument and in some embodiments overlay the graphical profile of temperature changes with the physical rendering 775 of the medical instrument.

Any of the determinations described above with respect to FIGS. 8-9 that are performed by the temperature sensing logic 198 may be displayed on the display 170 of the console 110. Additionally, any of the determinations described above may be provided to a clinician through alerts or notifications via the display 170 or via speakers (not shown). Additionally, the alerts or notifications may be transmitted to a network device, such as a mobile phone, a tablet, wearable technology, etc.

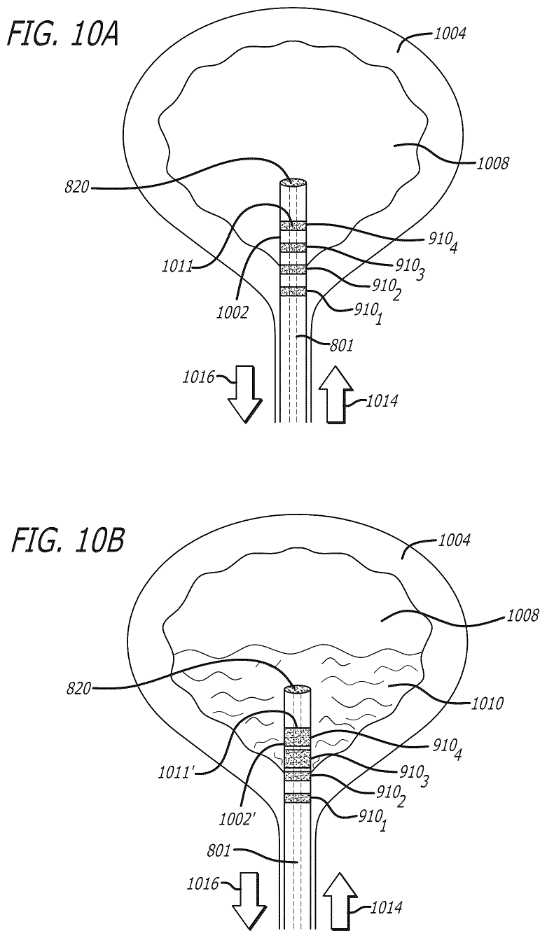

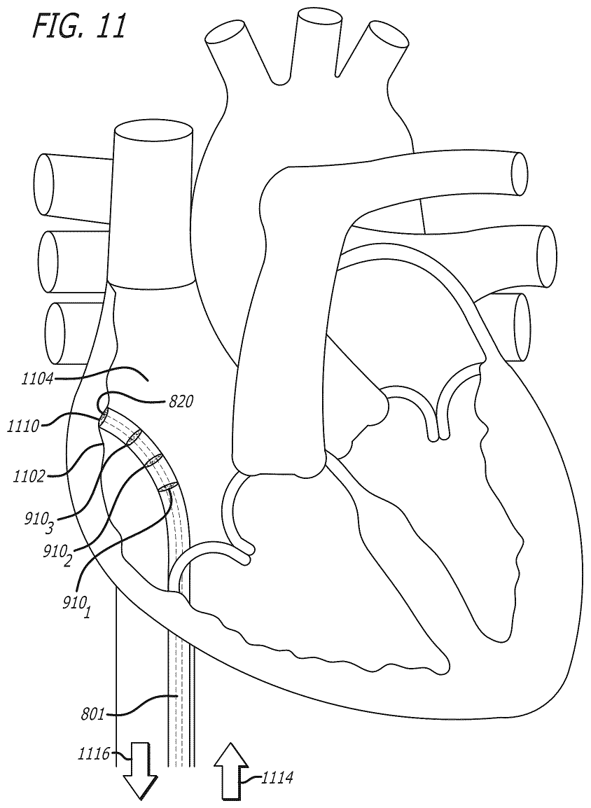

Referring now to FIGS. 10A-10B, exemplary embodiments of the medical instrument monitoring system of FIGS. 1A-1B deploying the medical instrument and optical fiber of FIG. 9 within a bladder of a patient are shown in accordance with some embodiments. The medical instrument 806 is shown with an optical fiber 801 disposed therein having a plurality of sensors 910 (e.g., sensors 910₁-910₄) distributed along a longitudinal length of the optical fiber 801. The distal tip of the medical instrument 806 is disposed within an interior portion 1008 of a bladder 1004 of a patient. The optical fiber 801 of medical instrument 806 is shown to be receiving an incident light signal 1014, and propagating a reflected light signal 1016 back to the console 110. In certain embodiments, each one of the plurality of sensors 910 is configured to reflect light of a prescribed spectral width, as discussed above with respect to at least FIGS. 8-9. As discussed above, the fiber optic temperature sensing system may determine a temperature of the bladder and analyze the determined temperature to determine whether the bladder contains urine.

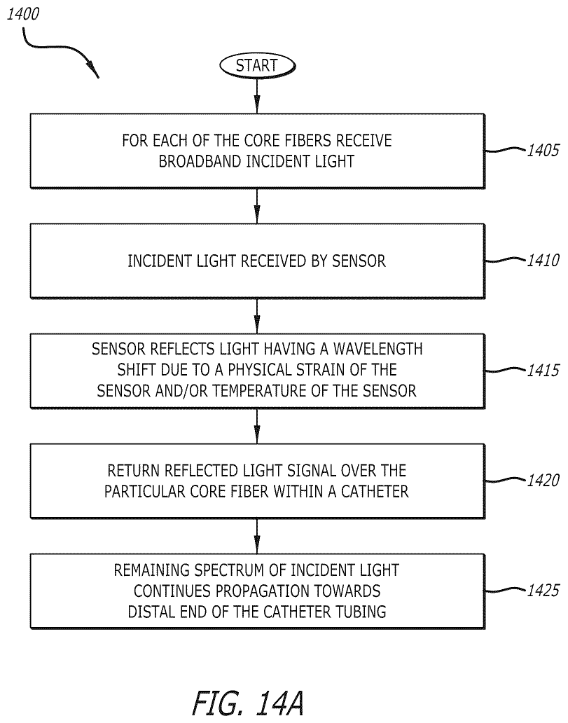

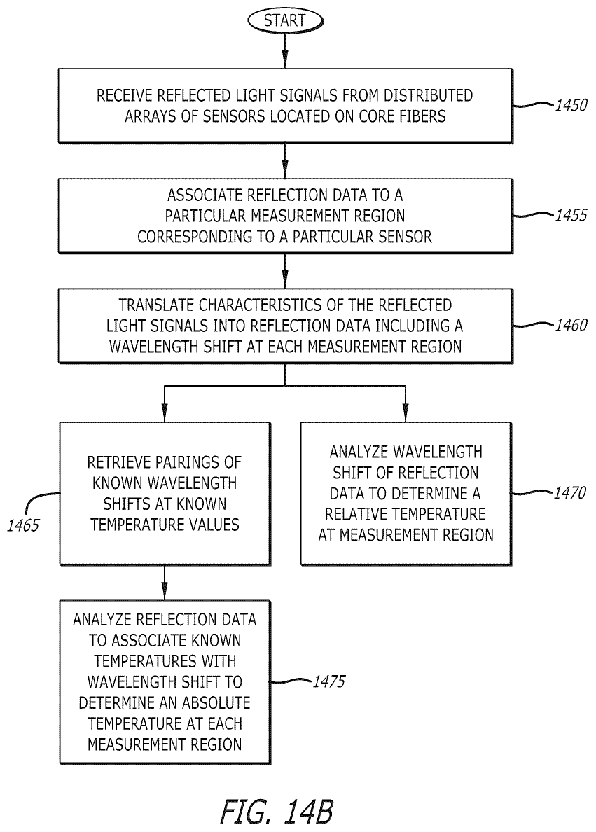

Referring to FIG. 10A, the interior portion 1008 of the bladder 1004 is shown to be empty, indicating the patient has recently urinated. An absence of urine may correspond to a lower temperature value, relative to a temperature of bladder having urine storage. For example, urinary bladder temperature may be influenced by either urine volume or urine flow rate. Based on the temperature of the bladder, the plurality of sensors 910 located within the optical fiber 801 are altered to sensor state 1011. As discussed, sensor states 1011 may correlate to a lower refractive index. In addition, the empty bladder temperature may induce a lower degree of thermal expansion in optical fiber 801 causing sensor spacings 1002 to be in a non-expanded state. Both the sensor states 1011 and sensor spacings 1002 may cause each of the plurality of sensors 910 to transmit a reflected light signal 1016 having a specific wavelength shift. The temperature sensing logic 198 then determines the temperature at each of the plurality of sensors 910, as discussed with respect to at least FIGS. 13-14. The determined temperature level may then be compared a known or expected value to determine whether urine is present. For example, a recorded temperature below a predefined value may indicate no urine is present in the bladder 1004 or that a urination has recently occurred.

Referring to FIG. 10B, the interior portion 1008 of the bladder 1004 has a store of urine 1010, indicating the patient has not recently urinated. The presence of urine 1010 in the bladder 1004 may cause a slight increase in ambient temperature of the interior portion 1008. The higher temperature of bladder 1004, corresponds to sensor state 1011′ having a higher refractive index than a sensor at a lower temperature (e.g., the empty bladder 1004). In addition, the optical fiber 801 undergoes a higher degree of thermal expansion at higher temperatures, resulting in sensor spacings 1002′. The temperature sensing logic 198 analyzes the reflected light signals to determine the temperature at each of the plurality of sensors 910. The determined temperature level may then be compared a known or expected value to determine whether urine is present. For example, a recorded temperature above a predefined value may indicate urine is present or that a urination has not recently occurred. It is contemplated that the determined temperature may indicate a specific volume of urine present in some embodiments.

In alternative embodiments, the temperature sensing system described above with respect to FIGS. 10A-10B may be implemented in other organs (e.g., stomach, liver, kidney, rectum, etc.). More specifically, the medical instrument 806 may be placed in a rectum of a patient, and the optic temperature sensing functionality may sense whether a bowel movement has occurred in the patient based on temperature fluctuations.

Referring now to FIG. 11, an embodiment of medical instrument monitoring system of FIGS. 1A-1B deploying the medical instrument and optical fiber of FIG. 9 in a heart of a patient during an ablation procedure is shown in accordance with some embodiments. The medical instrument 806 is shown with an optical fiber 801 disposed therein having a plurality of sensors 910 (e.g., sensors 910₁-910₄) distributed along a longitudinal length of the optical fiber. The distal tip of the medical instrument 806 is disposed within a right atrium 1104 of a patient at an ablation site 1110. As used herein, the term “ablation” generally refers to the removal and/or destruction of tissue or tissue function through the use of high or extremely low temperatures. In certain embodiments, a single medical instrument may perform both the ablation procedure and the optic temperature sensing. In an alternative embodiment, the medical instrument conducting the optic temperature sensing is seperate from the ablation instrument (e.g., ablation catheter).

During ablation, heart tissue at the ablation site 1110 may be heated or cooled to temperatures outside the range of a core body temperature of a patient. In certain embodiments, each one of the plurality of sensors 910 is configured to reflect light of a prescribed spectral width to determine the temperature at the ablation site 1110. Specifically, the distal sensor 820 may continuously monitor temperature fluctuations at the ablation site 1110 to provide boundary temperature feedback during ablation. More specifically, the ablation site 1110 and an adjacent tissue 1102 will experience higher temperatures during an ablation procedure. The plurality of sensors 910 may experience an alteration in their reflection characteristics based on the ambient temperature variations. In certain embodiments, the medical instrument monitoring system may provide a binary temperature feedback alert if the determined temperatures during the ablation procedure exceed a threshold range. Stated differently, if, during an ablation procedure, a temperature is recorded that is too hot or too cold compared to a known or expected temperature range of that procedure, the system may provide feedback to the user of the system in the form of real-time continuous feedback and/or an alert.

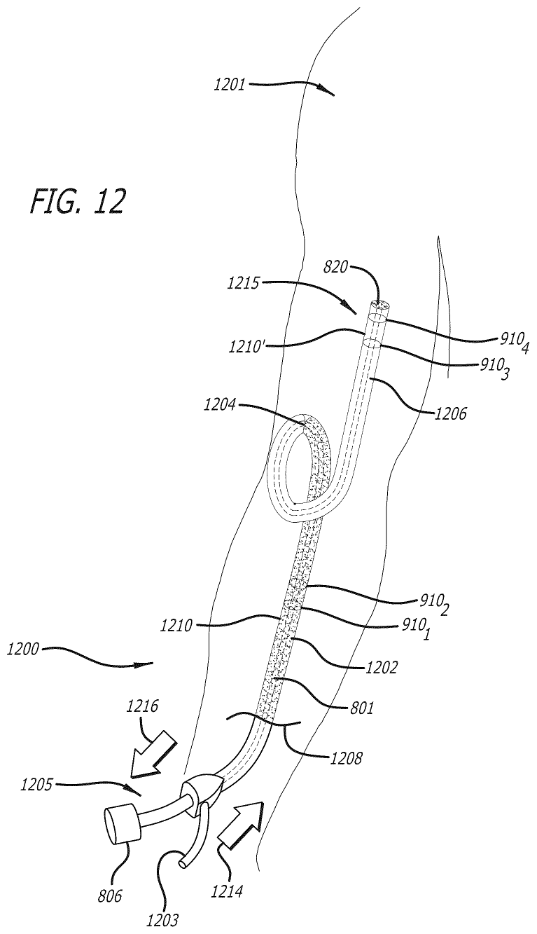

Referring now to FIG. 12, an exemplary embodiment of the medical instrument monitoring system of FIG. 1A during insertion and operation, is shown to be infusing a fluid in accordance with some embodiments. Herein, the medical instrument 806 generally includes integrated tubing with a proximal portion 1205 that generally remains exterior to the patient 1201 and a distal portion 1215 that generally resides within the patient 1201 after placement is complete, where the medical instrument 806 enters the patient 1201 at insertion site 1208. In certain embodiments, the medical instrument 806 may be an infusion catheter configured to intravenously infuse medicines, drugs, nutrition, and/or other fluids. It is contemplated that during infusion, the medical instrument infusion monitoring system 1200 may simultaneously perform other measurements or procedures (e.g., temperature sensing, oxygen sensing, heart chamber or blood vessel viewing, blood pressure monitoring, etc.). Once the distal tip of the medical instrument 806 is placed in the body of a patient 1201 at an infusion site proximal to the distal sensor 820, liquids (e.g., fluid 1202) may travel from an external source (not shown) through a bifurcation leg 1203 to the infusion site proximate the distal sensor through the medical instrument 806.

In certain embodiments, the integrated tubing of the medical instrument 806 includes the optical fiber 801 having one or more core fibers, with each core fiber including a plurality of sensors (e.g., sensors 910₁-910₄) spatially distributed along the length of the core fiber. As discussed with respect to at least FIG. 9, each of the plurality of sensors 910 is configured to receive an incident light signal 1214 and reflect light of different spectral width in the form of reflected light signal 1216. The temperature sensing logic 198 is configured to analyze the reflected light signal 1216 to determine a temperature at a location of each of the plurality of sensors 910. In certain embodiments, this optic temperature sensing system may be configured to determine an infusion status or an infusion error during infusion. Specifically, fluid 1202 is typically stored at a temperature range of 20-22° C. (e.g., “room” temperature) and may be infused into the patient 1201 at that temperature. However, an average core body temperature is approximately 37° C. The temperature sensing logic 198 is configured to determine the temperature at specific sensor locations and determine whether a fluid has infused past the specific locations based at least on the recorded temperatures.

Specifically, with continued reference to FIG. 12, the fluid 1202 traverses the length of the medical instrument until reaching sensor 910₁. As discussed above, the ambient temperature (e.g., temperature of the fluid 1202 at the location of sensor 910₁) may alter the characteristics of sensor 910₁, resulting in a specific wavelength shift in the reflected light signal 1216. The temperature sensing logic is configured to associate the reflected light signal 1216 with a first temperature measurement, as discussed with respect to at least FIGS. 9 and 14. The console 110 may compare the first temperature measurement with predefined parameters to determine an infusion status. For example, if the temperature at sensor 910₁ is within a predefined temperature range (e.g., 20-22° C.) the console 110 may indicate that infusion is progressing normally. Stated differently, if the temperature at sensor 910₁ is determined to be sufficiently close in value to the temperature that fluid 1202 is stored and infused at, the fluid has effectively infused to at least the portion of medical instrument 806 where sensor 910₁ is located.

As the fluid continues to traverse through the integrated tubing of the medical instrument 806, each of the plurality of sensors 910 may continuously provide temperature measurements to determine the infusion status in accordance with the methodology described above. In some embodiments, the medical instrument infusion monitoring system can be configured to detect an infusion error. For example, the fluid 1202 may continue to infuse through the length of the medical instrument 806 until reaching a blockage point 1204. The blockage point 1204 may be caused by a kink, occlusion, or other obstruction within the tubing, impeding the normal infusion flow of the fluid 1202. Since fluid 1202 cannot pass blockage 1204, end region 1206 of the medical instrument 806 will not contain fluid 1202 nor will fluid 1202 infuse into the patient at the distal infusion site proximate to the distal sensor 820. In this example, sensor 910₃ may detect a warmer temperature due to the end region 1206 being in equilibrium with the core temperature of a patient. If temperature sensing logic 198 determines the temperature at a sensor point is within a predefined temperature range (e.g., 36-38° C.), the console 110 may indicate an infusion error indicating that fluid 1202 has not infused to the portion of the medical instrument 806 where the sensor 910₃ is located. In some such instances, the incident light signal 1214 may propagate beyond the kink, occlusion, or other obstruction within the tubing, thus enabling detection of a temperature at a point distal to the kink, occlusion, or other obstruction. In other embodiments, the incident light signal 1214 may also be prevented from passing beyond the kink, occlusion, or other obstruction; thus, the lack of reflect light signals for a set of distal sensors, e.g., 910₃-910₄, would indicate a problem.

In some embodiments, the medical instrument infusion system 1200 may be configured to assist with determining a location of the infusion error. Specifically, after determining the temperature at each sensor, the logic of the console 110 may detect the specific location at which the temperature transitions from a colder value to a warmer value. For instance, as the fluid traverses through the integrated tubing of the medical instrument 806, it typically maintains a temperature that lower than a core body temperature. Each of the plurality of sensors 910 along the optical fiber 801 continuously monitors the temperature of the fluid. If an infusion error occurs, such as a blockage or leak, the temperature measured by the sensors will begin to shift towards core body temperature at the location of the error. The console 110 then uses the determined temperature data to pinpoint the specific location of the error.

Any of the determinations described above with respect to FIGS. 10-12 that are performed by the temperature sensing logic 198 may be displayed on the display 170 of the console 110. Additionally, any of the determinations may be provided to a clinician through alerts or notifications via the display 170 or via speakers (not shown). Additionally, the alerts or notifications may be transmitted to a network device, such as a mobile phone, a tablet, wearable technology, etc.

Referring now to FIG. 13, a flowchart illustrating an exemplary method of operations conducted by the medical instrument monitoring system of either of FIGS. 1A-1B deploying the medical instrument and optical fiber of FIG. 8 to achieve optic temperature sensing is shown in accordance with some embodiments. Each block illustrated in FIG. 13 represents an operation of the method 1300. It should be understood that not every operation illustrated in FIG. 13 is required. In fact, certain operations may be optional to complete aspects of the method 1300. Prior to initiation of the method 1300, it is assumed that a medical instrument having an optical fiber disposed therein is advancing through a body conduit of a patient.

According to one embodiment of the disclosure, for each core fiber, broadband incident light is supplied to propagate through a particular core fiber (block 1305). Unless discharged, the incident light is first received by a reference sensor positioned at a location external to the patient body. It should be understood that the reference sensor is positioned at a location with a known temperature. Light then continues to propagate along the core fiber until reaching a distal sensor located at a measurement region in the patient body. In certain embodiments, the distal sensor is configured to measure an ambient temperature of a patient body proximate the distal tip of the optical fiber. As discussed above, both the reference sensor and the distal sensor are configured to receive an incident signal and reflect light of a specified spectral range. The temperature sensing logic of the console first receives reflected light signal from the reference sensor (block 1310) and subsequently receives the reflected light signal from the distal sensor (1315).

Based on the reflected light signal from the reference sensor, the temperature sensing logic may determine a wavelength shift at a known external temperature (1320). For example, an external reference sensor may be designed to reflect a wavelength of 1550 nanometers (nm.). At an external temperature of 22° C., the reference sensor may be unaffected by temperature and reflect a wavelength of 1550 nm. (e.g., a net zero wavelength shift). However, it is contemplated that external reference sensor may be affected by slight external temperature changes and reflect a wavelength shifted to a higher value or lower value by specific magnitude. In either embodiment, the temperature sensing logic determines the wavelength shift and correlates the wavelength shift with the reference temperature (block 1320). The method continues with the temperature sensing logic determining the wavelength shift at the distal sensor from the received reflected light signal (block 1325).

Once the reference temperature, the reference wavelength shift, and the wavelength shift at the distal sensor are recorded, the temperature sensor may compare and analyze the reflected wavelength shifts at each of the reference sensor and distal sensor (block 1330). Specifically, the temperature sensing logic uses the previously described relationship between temperature and wavelength shift to determine the temperature at the distal sensor (block 1335). For example, the wavelength shift due to a change in temperature of 1° C. is approximately 10 picometers (pm.) for fused silica. With continuing reference to the previous example, and assuming a net zero wavelength shift by the external sensor positioned at 22° C., a positive 90 pm. wavelength shift in reflected light signal transmitted by the distal sensor correlates to a temperature of approximately 31° C. It is contemplated that other processes and procedures may be performed to utilize the wavelength shifts as measured by sensors along each of the core fibers to render appropriate changes in the temperature of the patient body.

FIGS. 14A-14B are flowcharts illustrating exemplary methods of operations conducted by the medical instrument monitoring system of either of FIGS. 1A-1B deploying the medical instrument and optical fiber of FIG. 9 to achieve optic temperature sensing are shown in accordance with some embodiments. Herein, the medical instrument includes an optical fiber disposed therein having one or more core fibers. Furthermore, each core fiber includes a plurality of sensors spatially distributed along its length between at least the proximal and distal ends of the medical instrument. The plurality of sensors are distributed to position sensors at different regions of the core fiber to enable distributed measurements of temperature throughout the entire length or a selected portion of the medical instrument. These distributed measurements may be conveyed through reflected light of different spectral widths (e.g., specific wavelength or specific wavelength ranges) that undergoes certain wavelength shifts based on the temperature changes.

According to one embodiment, as shown in FIG. 14A, for each core fiber, broadband incident light is supplied to propagate through a particular core fiber (block 1405). Unless discharged, upon the incident light reaching a sensor of a distributed array of sensors, light of a prescribed spectral width associated with the first sensor is to be reflected back to an optical receiver within a console (block 1410). The sensor transmits a light signal which may include a change (shift) in the wavelength of the reflected light compared to the wavelength of the incident light (block 1415). The wavelength shift may be caused by either a physical strain on the sensor (e.g., curvature of the medical instrument, discussed above with respect to at least FIG. 6B) or the temperature of the sensor. The sensor returns the reflected light signal over the core fiber and the remaining spectrum of the incident light continues propagation through the core fiber toward a distal end of the medical instrument (blocks 1420-1425). The remaining spectrum of the incident light may encounter other sensors of the distributed array of sensors, where each of these sensors would operate as set forth in blocks 1405-1420 until the last sensor of the distributed array of sensors returns the reflected light signal associated with its assigned spectral width and the remaining spectrum is discharged as illumination.

Referring now to FIG. 14B, during operation, multiple reflected light signals are returned to the console from each of the plurality of core fibers residing within the a medical instrument, such as the catheter of FIG. 1B (block 1450). The optical receiver may optionally associate the received reflection data to a particular measurement region (e.g., location of each sensor (block 1455). In particular, the optical receiver receives reflected light signals from the distributed arrays of sensors located on the center core fiber and the outer core fibers and translates the reflected light signals into reflection data, namely electrical signals representative of the reflected light signals (block 1460). The reflection data may include wavelength shifts caused by a physical strain and/or temperature of the sensor at each measurement region.

The temperature sensing logic may then analyze the reflection data wavelength shift to determine a relative temperature at each measurement region (block 1470). Specifically, each sensor will transmit a reflected light signal having a wavelength shift. The difference in wavelength shift from one sensor to the next can be correlated to determine a relative temperature change. For example, if the first sensor has a wavelength shift of 10 pm., and the second sensor has a wavelength shift of 20 pm., the temperature sensing logic may determine that the second sensor is disposed at a region approximately 1° C. hotter than the region of the first sensor. Stated different, the wavelength shifts will indicate how hot or cold one measurement region is with respect to a different or subsequent measurement region.

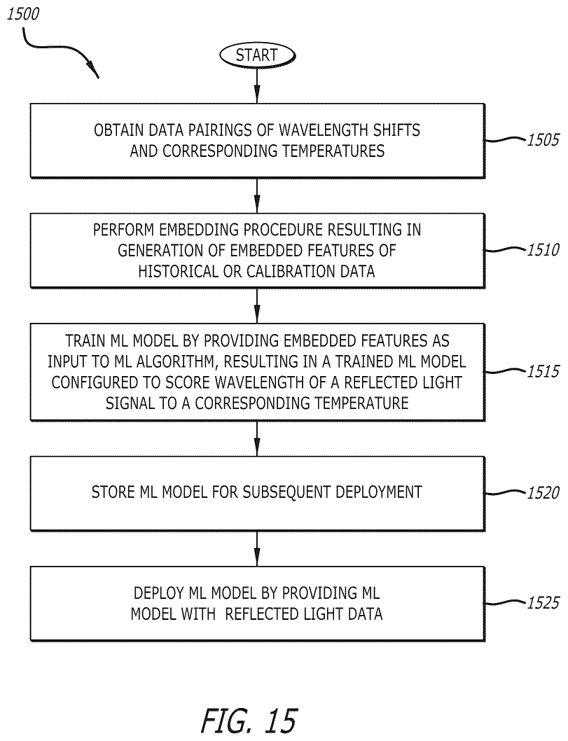

In alternative embodiments, the optic temperature sensing method described above with respect to FIGS. 14A-14B may include determining the temperature at a sensing point based on heuristics or run-time analytics. In this embodiment, the temperature sensing logic may retrieve pairings of known wavelength shifts at known temperature values (block 1465). From this data store, the reflection data may be analyzed to associate the wavelength shift with an absolute temperature at each measurement region (block 1475). For example, the ML-based logic 199 within the temperature sensing logic 198 may be configured in accordance with machine-learning techniques to access a data store (library) with pre-stored data pertaining to different temperatures in which the distal sensor experienced similar or identical wavelength shifts. From the pre-stored data, the current temperature of the medical instrument at each of the plurality of sensors may be determined, as described below with respect to at least FIG. 15. It is contemplated that other processes and procedures may be performed to utilize the wavelength shifts as measured by sensors along each of the core fibers to render appropriate changes in the temperature of the patient body.

It some embodiments, the optic temperature sensing methods described above with respect to FIGS. 14-15 may include determining whether wavelength shifts are caused by a physical strain or a temperature. It is contemplated that the temperature sensing logic 198 may work in conjunction with the shape sensing logic 194 to isolate whether a wavelength shift is due to thermal effects or mechanical deformation. This may include positioning the plurality of sensors 910 on a neutral axis to eliminate the effects of physical strain, thereby ensuring that any wavelength shifts are caused solely by temperature changes. Alternatively, a second sensor may be used to differentiate between the two types of shifts.

FIG. 15 is a flowchart illustrating an embodiment of training and deploying a machine learning model to perform optic temperature sensing in accordance with some embodiments. Each block illustrated in FIG. 15 represents an operation of the method 1500. It should be understood that not every operation illustrated in FIG. 15 is required. In fact, certain operations may be optional to complete aspects of the method 1500. The operations of the method 1500 may be performed by, for example, the temperature sensing logic 198 and the ML-based logic 199 of FIGS. 1A-1B.

The method 1500 begins with the operation of parsing historical or known reflected wavelength signals to obtain data pairings of wavelength shifts and corresponding temperatures (block 1505). The extraction of wavelength shifts and corresponding temperatures may be part of a feature extraction process, where each form at least a portion f a feature set that will be analyzed by a machine learning (ML) algorithm as training data as discussed below. Each sensor embedded within the optical fiber is designed to reflect light of a different, specific spectral width. In certain embodiments, a calibration process can be implemented to collect data of reflected light signals at specified temperatures. For example, reflected light signals for each sensor may be recorded at incremented temperature intervals (e.g., every 0.1 degrees over a predefined temperature range). Thus, the feature extraction process includes identifying a sensor, identifying the expected reflected wavelength for the sensor (which may be stored in a database along with the historical data, e.g., the database storing the reflect data 192 as shown in FIGS. 1A-1B, such as in a separate key-value pair table or as metadata of the historical data), identifying the wavelength shift (the change in received wavelength relative to the expected wavelength), and identifying a corresponding temperature. The corresponding temperature may also be stored in a separate key-value pair table or as metadata.

The method 1500 continues with the operation of performing an embedding procedure resulting in generation of embedded features of historical data (block 1510). For example, original, historical data, which may include one or more of a sensor location within the medical instrument, temperature value, an incident light signal, an incident light wavelength, a reflected light signal, or a reflected light wavelength, or a wavelength shift, may converted into a feature vector. For instance, the embedding procedure may be a one-hot encoding of the data; however, other embedding procedures may be utilized. The embedded features such as the correlation between temperature and reflected light signal, may be stored in a data store. Once historical data has been collected, and mapped to a feature vector, an ML model can be trained by providing embedded features as input to an ML algorithm for training (block 1515). The training may include initializing the parameters of the ML model, e.g., using a default set of parameters, providing a batch of the historical data within the feature vector as input to the ML model, which predicts a temperature for data points within the batch (e.g., a temperature for each wavelength shift), with the prediction compared to the actual temperature to compute a loss value. A step of backpropagation is performed to determine the gradients (partial derivative of the loss value with respect to each parameter of the model) and a gradient step process is performed to update the parameters (e.g., weights and biases). Additional batches are then provided as input to the ML model with the steps above repeated into order to continue to adjust the model parameters. A number of epochs may be performed, which represents a number of times the training data is processed. The ML model can be stored for subsequent deployment (block 1520).

The ML model is then deployed by providing the model with subsequently received reflected light data (1525). Deployment of the ML model results in a prediction of a temperature with respect to a wavelength shift (and optionally, in accordance with other features provided as input and as provided as training data features including, a sensor location within the medical instrument, temperature value, an incident light signal, an incident light wavelength, a reflected light signal, or a reflected light wavelength, etc.). In some instances, the results of the ML model are provided as part of a graphical user interface (GUI), such as that shown in FIG. 7. In some instances, the temperatures may be illustrated as a line graph with temperature readings vertically aligned with sensors on a graphical rendering of the medical instrument. In other instances, the temperatures may be provided as a listing and/or stored for future training, re-training, or fine-tuning of the ML model.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical instrument system for determining a temperature within a patient body, the system comprising:
a medical instrument comprising an optical fiber having one or more core fibers having at least a first sensor; and
a console including one or more processors and a non-transitory computer-readable medium having stored thereon logic which, when executed by the one or more processors, is configured to cause operations including:
providing an incident light signal to the optical fiber;
receiving a reflected light signal from the first sensor wherein the first sensor is configured to reflect light of a first wavelength;
determining a first wavelength shift of the reflected light signal wherein the first wavelength shift is caused by the temperature at the first sensor; and
processing the reflected light signal to determine the temperature within the patient body at a location of the first sensor through (i) correlating the first wavelength shift with a wavelength shift at other sensor locations or (ii) correlating the first wavelength shift with the temperature known to cause the first wavelength shift.

2. The system according to claim 1, wherein the first sensor is disposed at a distal tip of the optical fiber.

3. The system according to claim 2, wherein the processing of the reflected light signal involves a comparison between a wavelength shift of the reflected light signal with a wavelength shift of a reference sensor corresponding to a known external reference temperature.

4. The system according to claim 1, wherein each of the one or more core fibers includes a plurality of sensors distributed along a longitudinal length of a corresponding core fiber.

5. The system according to claim 4, wherein the logic, when executed by the one or more processors, causes further operations including generating a temperature gradient display.

6. The system according to claim 1, wherein the logic which, when executed by the one or more processors, is configured to cause further operations including determining a location of the distal tip of the optical fiber within the patient body at least based on the temperature.

7. The system according to claim 1, wherein the reflected light signal indicates aspiration within the patient.

8. The system according to claim 1, wherein the medical instrument is located within a bladder of the patient body, and wherein the reflected light signal indicates urination has recently occurred.

9. The system according to claim 1, wherein the medical instrument is located at an ablation site, and wherein the reflected light signal indicates a temperature at the ablation site.

10. The system according to claim 1, wherein the medical instrument is configured to infuse a fluid, and wherein the reflected light signal indicates an infusion error.

11. A method for determining a temperature within a patient body, the method comprising:
providing an incident light signal to an optical fiber;
receiving a reflected light signal from the first sensor wherein the first sensor is configured to reflect light of a first wavelength;
determining a first wavelength shift of the reflected light signal wherein the first wavelength shift is caused by the temperature at the first sensor; and
processing the reflected light signal to determine the temperature within the patient body at a location of the first sensor through (i) correlating the first wavelength shift with a wavelength shift at other sensor locations or (ii) correlating the first wavelength shift with the temperature known to cause the first wavelength shift.

12. The method according to claim 11, wherein the first sensor is disposed at a distal tip of the optical fiber.

13. The method according to claim 12, wherein the processing of the reflected light signal involves a comparison between a wavelength shift of the reflected light signal with a wavelength shift of a reference sensor corresponding to a known external reference temperature.

14. The method according to claim 11, wherein each of the one or more core fibers includes a plurality of sensors distributed along a longitudinal length of a corresponding core fiber.

15. The method according to claim 14, further comprising generating a temperature gradient display.

16. The method according to claim 11, further comprising determining a location of the distal tip of the optical fiber within the patient body at least based on the temperature.

17. The method according to claim 11, wherein the reflected light signal indicates aspiration within the patient.

18. The method according to claim 11, wherein the medical instrument is located within a bladder of the patient body, and wherein the reflected light signal indicates an urination has occurred.

19. The method according to claim 11, wherein the medical instrument is located at an ablation site, and wherein the reflected light signal indicates a boundary temperature at the ablation site.

20. The method according to claim 11, wherein the medical instrument is configured to infuse a substance, and wherein the reflected light signal indicates an infusion error.
